(19) United States
(12) Patent Application Publication
(10) Pub. No.: US 2025/0325572 A1
(43) Pub. Date: Oct. 23, 2025

(54) TREATMENTS FOR PAIN

(71) Applicant: Sevenless Therapeutics Limited, Canterbury Kent (GB)

(72) Inventors: Neil BENSON, Canterbury Kent (GB); Mark Alasdair NAYLOR, Canterbury Kent (GB)

(21) Appl. No.: 19/170,881

(22) Filed: Apr. 4, 2025

Related U.S. Application Data
(63) Continuation of application No. PCT/US2023/052567, filed on Oct. 4, 2023.

(30) Foreign Application Priority Data
Oct. 5, 2022 (GB) 2214652.6
Oct. 6, 2022 (GB) 2214722.7

(51) Int. Cl. A61K 31/675; A61K 31/502; A61K 31/5025; A61K 31/517; A61K 31/519; A61K 31/5377; A61K 31/553; A61P 29/02

(52) U.S. Cl. CPC A61K 31/675; A61K 31/502; A61K 31/5025; A61K 31/517; A61K 31/519; A61K 31/5377; A61K 31/553; A61P 29/02

(57) ABSTRACT

The application describes suitable compounds for the treatment of pain and their use in combination with known pain medications.

This application is a bypass continuation of International (PCT) Application No. PCT/GB2023/052567, filed Oct. 4, 2023, which claims the benefit of priority of Great Britain patent application serial no. 2214722.7, filed on Oct. 6, 2022, Great Britain patent application serial no. 2214722.6, filed on Oct. 5, 2022, which are incorporated by reference herein in their entireties.

The present invention describes the use of compounds that bind to the Son of Sevenless homolog 1 receptor (SOS1) protein thereby inhibiting a cascade pathway, leading to a reduction in pain.

There remains a need for novel treatments for the treatment of pain. Many of the most efficacious and frequently prescribed pain treatments are opioids. These drugs have a high potential for abuse and addiction. However, to date there are no treatments of comparable efficacy to replace them as front-line treatments. To provide a paradigm shift in the treatment of pain requires the identification of new targets and pathways within the body.

This application describes the identification and exploitation of SOS-Ras in a suitable pathway for the treatment of Pain.

SOS1 inhibitors have recently been identified capable of mediating several conditions:

WO2019/122129 describes benzylamino substituted pyridopyrimidines as SOS1 inhibitors useful in the treatment of cancerous growth in oncology.

WO2018/115380 describes benzylamino substituted quinazolines as SOS1 inhibitors, similarly useful in the treatment of cancerous growth in oncology.

WO2018/172250 describes a genus of 2 methyl quinazolines for use in treating hyper-proliferative diseases.

WO2019/201848 describes a further genus of 2 methyl quinazolines for use in treating hyper-proliferative diseases.

WO2020/173935 teaches new isoindolinone substituted indoles as RAS inhibitors.

Further SOS1 inhibitors are taught in Proceedings of the National Academy of Sciences of the United States of America (2019), 116(7), 2551-2560.

Various RAS inhibitors including a subset of Ras inhibitors known as KRAS have also recently been identified. WO2018/068017, WO2018/140513, WO2018/140514 & WO2020/173938 all teach new compounds with activity as RAS inhibitors to treat cancer. A summary of new Ras inhibitors and their clinical status may be found at RSC Med. Chem., 2020, 11, 760.

Surprisingly it has now been found that the SOS1/Ras pathway can be exploited for the treatment of pain.

Accordingly, the present invention provides a series of known SOS1 inhibitors for use in the treatment of Pain.

### FIGURES

FIG. 1. NGF signal transduction pathway leading to pain and the clinical drugs that validate the pathway. NGF binds to TrkA and subsequent signal transduction culminates in the nuclear accumulation of diphopshorylated Extracellular signal-regulated kinase (dppERKnuc) in neurons, upregulating pain genes.

FIG. 2. Clinical genetic validation of the target. In NF1, patients have a mutation in the neuronal gap protein (NF1). The mutation causes a loss of function, preventing the normal turnover of GTP on RAS_{GTP} to GDP in turn increasing the concentration of RAS_{GTP} and leading to excess signalling, tumours and pain.

### SUMMARY OF INVENTION

This application describes the identification and exploitation of the SOS-Ras target as appropriate pathways for the treatment of Pain.

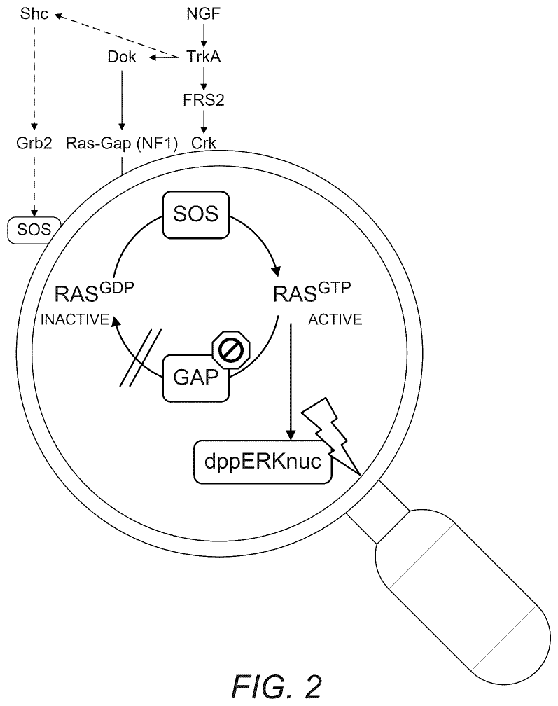

Ras proteins are known to be a key element in the maintenance of tumours and so the target has long been considered attractive in oncology. However, until recently SOS1-Ras was seen as an undruggable target. The canonical property of Ras is that of a small GTPase which normally cycles between a GTP-bound active state and a GDP-bound inactive state, facilitated in part by GTPase activating protein (GAP) stimulation of GTP hydrolysis (FIG. 2). However, when Ras proteins are mutationally activated, impaired GAP stimulation favours the formation of persistently GTP-bound Ras. This critical biochemical defect prompted the earliest efforts to target mutant Ras. By analogy to the ATP-competitive inhibitors that are effective antagonists of protein kinases, identification of GTP-competitive inhibitors of Ras has been attempted. However, whereas ATP binds protein kinases with low micromolar affinity, GTP binds Ras proteins with picomolar affinity, preventing discovery of effective inhibitors.

With the discovery of suitable SOS1 inhibitors it has been possible to investigate the pathway for additional indications, other than cancer. Surprisingly, detailed investigation has now revealed its possible to have a positive effect on pain by using known SOS1 inhibitors.

The present invention provides SOS1 inhibitors for use in the treatment of Pain.

Suitable SOS1 inhibitors include those disclosed in:
WO/2022/187266 NOVEL SUBSTITUTED BICYCLI AZA-HETEROCYCLES AS SOS1 INHIBITORS, WO/2022/187236 NOVEL SUBSTITUTED TRICYCLIC AZA-HETEROCYCLES AS SOS1 INHIBITORS, WO/2022/184116 NEW SOS1 INHIBITOR, PREPARATION METHOD THEREFOR AND USE THEREOF, WO/2022/170952 POLYCYCLIC PYRIDAZINONE DERIVATIVE SERVING AS SOS1 INHIBITOR, PREPARATION METHOD THEREFOR AND USE THEREOF, WO/2022/170802 PYRIMIDO-PYRIDONE DERIVATIVE AS SOS1 INHIBITOR, PREPARATION METHOD THEREFOR AND USE THEREOF, WO/2022/170917 POLYCYCLIC PYRIMIDINE DERIVATIVE AS SOS1 INHIBITOR, AND PREPARATION METHOD THEREFOR AND USE THEREOF, WO/2022/171018 SUBSTITUTED BENZOPYRIMIDINE OR PYRIDOPYRIMIDINE AMINE INHIBITOR, AND PREPARATION METHOD THEREFOR AND USE THEREOF, WO/2022/171184 HETEROCYCLIC COMPOUND AS SOS1 INHIBITOR, WO/2022/166974 PYRIDOPYRIMIDINONE DERIVATIVE, PREPARATION METHOD THEREFOR, AND USE THEREOF, WO/2022/166592 SUBSTITUTED PYRIMIDOPYRIDONE INHIBITOR, AND PREPARATION METHOD THEREFOR AND USE THEREOF, WO/2022/161480 SUBSTITUTED BICYCLO-AROMATIC HETEROCYCLIC AMINE INHIBITOR, PREPARATION METHOD THEREFOR, AND USE THEREOF, WO/2022/160931 PYRIDOPYRIMIDINE DERIVATIVE, PREPARATION METHOD THEREFOR AND USE THEREOF, WO/2022/161461 SOS1 INHIBITOR, PREPARATION METHOD THEREFOR, AND APPLICATION THEREOF, WO/2022/157629 PHARMACEUTICAL COMBINATIONS OF SOS1 INHIBITORS FOR TREATING AND/OR PREVENTING CANCER, WO/2022/156792 HETEROCYCLIC COMPOUNDS AS SOS1 INHIBITORS, WO/2022/148442 6-SUBSTITUTED PHOSPHORYL QUINAZOLINE DERIVATIVE, PREPARATION METHOD THEREFOR AND USE THEREOF, WO/2022/143533 QUINAZOLINE DERIVATIVE AND USE THEREOF IN MEDICINE, WO/2022/146698 SOS1 INHIBITORS AND USES THEREOF, WO/2022/135610 TETRACYCLIC COMPOUND, PHARMACEUTICAL COMPOSITION THEREOF AND USE THEREOF, WO/2022/140427 SOS1 INHIBITORS AND USES THEREOF, WO/2022/139304 NOVEL QUINAZOLINE DERIVATIVE COMPOUND AS SOS1 INHIBITOR, AND USE THEREOF, WO/2022/121813 SOS1 INHIBITOR, PHARMACEUTICAL COMPOSITION COMPRISING SAME, AND USE THEREOF, WO/2022/105921 PYRIMIDO-HETEROCYCLIC COMPOUND, PREPARATION METHOD THEREFOR, AND USE THEREOF, WO/2022/083657 SUBSTITUTED BENZO OR PYRIDOPYRIMIDINE AMINE INHIBITOR, PREPARATION METHOD THEREFOR, AND APPLICATION THEREOF, WO/2022/060583USE OF SOS1 INHIBITORS TO TREAT MALIGNANCIES WITH SHP2 MUTATIONS, WO/2022/058344 PYRIDO[2,3-D]PYRIMIDIN-4-AMINES AS SOS1 INHIBITORS, WO/2022/028506 SOS1 INHIBITOR, PHARMACEUTICAL COMPOSITION CONTAINING SAME, AND USE THEREFOR, WO/2022/026465 SOS1 INHIBITORS, WO/2022/017339 FUSED PYRIDAZINE DERIVATIVE, PREPARATION METHOD THEREFOR AND PHARMACEUTICAL USE THEREOF, WO/2021/249519 PYRIDINE-PYRIMIDINE DERIVATIVE, PREPARATION METHOD THEREFOR AND PHARMACEUTICAL USE THEREOF, WO/2021/249475 FUSED QUINAZOLINE DERIVATIVE, PREPARATION METHOD THEREFOR AND APPLICATION THEREOF IN MEDICINE.

Suitable SOS1 inhibitors include those described in the geni below:
(a) Wo2022/017339

A compound represented by the general formula (I) or its tautomer, meso, racemate, enantiomer, diastereomer, or its mixture form, or its Medicinal salt: in:

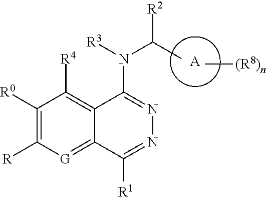

Ring A is aryl or heteroaryl;
G is CR5 or N atom;
R0 is selected from halogen, alkyl, alkoxy, haloalkyl, haloalkoxy, hydroxyalkyl, alkenyl, alkynyl, hydroxy, amino, —(CH2)pNR6R7, cycloalkyloxy, heterocyclyloxy, cycloalkyl, heterocyclyl, aryl and heteroaryl, wherein said alkyl, cycloalkyloxy, heterocyclyloxy, cycloalkyl, heterocyclyl, aryl and heteroaryl are each independently optionally selected from halogen, alkyl, haloalkyl, hydroxy, hydroxyalkyl, alkoxy, haloalkoxy, oxo, ═NH, amino, nitro, cyano, —S(O)2R9, —C(O)R10, cycloalkyl, heterocyclyl, aryl and heteroaryl are substituted with one or more substituents;
R1 is selected from hydrogen atom, halogen, alkyl, haloalkyl, alkoxy, haloalkoxy, hydroxyalkyl, cyano and cycloalkyl;
R2 is selected from halogen, alkyl, haloalkyl, hydroxyalkyl, hydroxy, cyano, cycloalkyl and heterocyclyl, wherein each of said alkyl, cycloalkyl and heterocyclyl is independently optionally selected Substituted from one or more substituents of halogen, alkyl, haloalkyl, hydroxy, hydroxyalkyl, alkoxy, haloalkoxy, amino, nitro and cyano;
R3 is selected from hydrogen atom, alkyl, haloalkyl, hydroxyalkyl, cycloalkyl, heterocyclyl, aryl and heteroaryl, wherein said alkyl, haloalkyl, hydroxyalkyl, cycloalkyl, hetero Cyclic, aryl, and heteroaryl are each independently optionally selected from halogen, alkyl, alkenyl, alkynyl, alkoxy, haloalkyl, haloalkoxy, cyano, amino, nitro, hydroxy, substituted with one or more substituents of hydroxyalkyl, cycloalkyl, heterocyclyl, aryl and heteroaryl;
R4 is selected from hydrogen atom, halogen, alkyl, haloalkyl, alkoxy, haloalkoxy, hydroxy, hydroxyalkyl and —(CH2)pNR6R7;
R and R5 are the same or different, each independently selected from hydrogen atom, halogen, alkyl, alkoxy, haloalkyl, haloalkoxy, alkenyl, alkynyl, cycloalkyl, heterocyclyl, aryl, heteroaryl group, cycloalkyloxy, heterocyclyloxy, aryloxy, heteroaryloxy, —(CH2)pNR6R7, cyano and nitro, wherein said alkyl, alkenyl, alkynyl, Cycloalkyl, heterocyclyl, aryl and heteroaryl are each independently optionally selected from alkyl, haloalkyl, alkoxy, haloalkoxy, halogen, cyano, nitro and —(CH2)qNR11R12 substituted by one or more of the substituents in;
R8 is the same or different, each independently selected from halogen, alkyl, alkenyl, alkynyl, haloalkyl, alkoxy, haloalkoxy, cyano, amino, —(CH2)pNR6R7, nitro, hydroxy, hydroxyalkane Alkyl, —S(O)2 alkyl, cycloalkyl, heterocyclyl, aryl and heteroaryl, wherein said alkyl, haloalkyl, hydroxyalkyl, cycloalkyl, heterocyclyl, aryl and heteroaryl are each independently optionally selected from hydroxy, halogen, haloalkyl, alkoxy, haloalkoxy, cyano, nitro, hydroxyalkyl, —(CH2)qNR11R12, cycloalkyl, heterocycle substituted with one or more substituents in aryl, aryl and heteroaryl;
R9 and R10 are the same or different, each independently selected from hydrogen atom, alkyl, haloalkyl, hydroxyalkyl, —(CH2)qNR11R12, cycloalkyl and heterocyclyl, wherein said alkyl, cycloalkyl and The heterocyclyl groups are each independently optionally substituted with one or more substituents selected from the group consisting of hydroxy, halo, alkyl, haloalkyl, hydroxyalkyl, alkoxy, haloalkoxy, cyano, amino, and nitro;
R6, R7, R11 and R12 are the same or different and are each independently selected from hydrogen atoms, alkyl groups, haloalkyl groups, hydroxyalkyl groups, cycloalkyl groups, heterocyclyl groups, aryl groups and heteroaryl groups;
p and q are the same or different, each independently selected from 0, 1 and 2;
n is selected from 0, 1, 2, 3, 4 and 5.
   (b) WO2022/105921

A pyrimido heterocyclic compound as shown in general formula I, or a pharmaceutically acceptable salt thereof, or its enantiomer, diastereomer, tautomer, twist isomer, solvates, polymorphs or prodrugs,

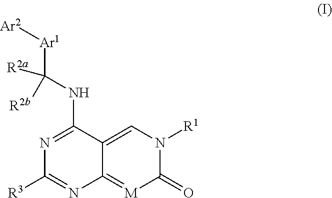

where:
R1 is independently selected from C1-C6 alkyl, C1-C6 haloalkyl, C2-C6 alkenyl, C2-C6 alkynyl, C3-C12 cycloalkyl, C4-C12 cycloalkenyl, 3-12 membered heterocycloalkane base, 5-12-membered aryl or 5-12-membered heteroaryl, carbocyclic or heteroatom-containing spiro/bridged/fused ring, wherein the C1-C6 alkyl, C2-C6 alkenyl, C2-C6 alkynyl, C3-C12 cycloalkyl, C4-C12 cycloalkenyl, 3-12 membered heterocycloalkyl, 5-12 membered aryl or 5-12 membered heteroaryl, carbocyclic or heterocyclic The spiro/bridged/fused ring of atoms can be optionally substituted by 1-3 Rn; or the above two Rn can form 3-12-membered saturated or partially unsaturated, or aromatic through carbon chains or heteroatoms Ring system; said Rn is selected from hydrogen, deuterium, halogen, cyano, nitro, amide, sulfonamide, hydroxyl, amino, urea, phosphoryl, alkyl phosphoroxy, alkylsilyl, C1-C6 Alkyl, C1-C6 alkoxy, haloalkyl, haloalkoxy, C1-C6 monoalkylamino, C1-C6 dialkylamino, alkenyl, alkynyl, 3-8 membered cycloalkyl or heterocycloalkane base, C1-C6 alkyl-S—, C1-C6 alkyl-SO—, C1-C6 alkyl-SO2—;
R2a and R2b are each independently selected from hydrogen, deuterium, halogen, C1-C6 alkyl, 3-8 membered cycloalkyl or heterocycloalkyl; and R2a and R2b or the substituent Rm on R2a and Ar may pass through a carbon chain Or heteroatoms form a 3-6 membered saturated or partially unsaturated or unsaturated ring system;
R3 is H, deuterium, halogen, hydroxyl, amino, cyano, C1-C6 alkyl, C1-C6 haloalkyl, C1-C6 alkoxy, C1-C6 haloalkoxy, C1-C6 alkylamino, 3-8-membered cycloalkyl or heterocycloalkyl, C2-C4 alkenyl, C2-C4 alkynyl, 5-10-membered aromatic ring or aromatic heterocyclic group;
M is independently selected from N or CR4, and R4 is selected from hydrogen, deuterium, halogen, cyano, C1-C6 alkyl, 3-8 membered cycloalkyl or heterocycloalkyl;
Ar1 and Ar2 are independently selected from 5-12-membered monocyclic or bicyclic aryl or heteroaryl groups, which may be substituted by one or more groups Rm selected from the group consisting of hydrogen: deuterium, halogen, cyano, nitro, substituted or unsubstituted amide, substituted or unsubstituted sulfonamide, hydroxyl, amino, urea, phosphoryl, alkylphosphooxy, alkylsilyl, C1-C10 Alkyl, C1-C10 alkoxy, C1-C10 alkoxyalkyl, C1-C10 haloalkyl, C1-C10 haloalkoxy, C1-C10 haloalkoxyalkyl, C1-C10 monoalkylamino, C1-C10 dialkylamino, C1-C10 monoalkylaminoalkyl, C1-C10 dialkylaminoalkyl, C1-C10 alkenyl, C1-C10 alkynyl, 3-12 membered cycloalkyl or heterocycloalkane base, 3-12 membered cycloalkyl or heterocycloalkylalkyl, C1-C10 alkyl-S—, C1-C10 alkyl-SO—, C1-C10 alkyl-SO2—, substituted or unsubstituted 5-12-membered aryl or heteroaryl, or the above two Rm can form a 3-12-membered saturated or partially unsaturated, or aromatic ring system through carbon chains or heteroatoms;
One or more hydrogen atoms on any of the above-mentioned groups can be substituted by a substituent selected from the following group: including but not limited to deuterium, halogen, C1-C3 alkyl, 3-6 membered cycloalkyl or heterocycloalkane wherein, the heteroaryl group contains 1-3 heteroatoms selected from the following group: N, O, P or S, and the heterocycloalkyl group contains 1-3 heteroatoms selected from the following group: N, O, P or S, the ring system includes spiro, bridged, fused, and saturated or partially unsaturated ring systems.
(c) WO2022/148442

6-substituted phosphorylquinazoline derivatives represented by formula I,

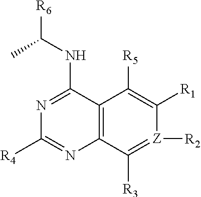

their tautomers, stereoisomers, hydrates, solvates, pharmaceutically acceptable salts or prodrugs:
in,
Z is a carbon atom or a nitrogen atom; and when Z is a nitrogen atom, R2 is absent;
R1 is
R11 and R12 are each independently C1-C6 alkyl or C1-C6 alkoxy; the C1-C6 alkyl or the C1-C6 alkoxy is independently substituted by one or more R13, the R13 is a substituent selected from the following: hydroxyl, amino, nitro, halogen, cyano; when there are multiple substituents, the substituents are the same or different;
Or, R11, R12 together with the phosphorus atom they are attached to form a substituent wherein, ring B is a 4-8 membered carbocyclic ring, a 4-8 membered alkene ring or a 4-8 membered heterocyclic ring;
Ra is independently hydrogen or a substituent selected from the group consisting of hydroxyl, amino, nitro, halogen, cyano, C1-C6 alkyl, 3-8 membered cycloalkyl; the C1-C6 alkyl, or the The 3-8 membered cycloalkyl groups are independently substituted by one or more Rf; when there are multiple substituents, the Rf is the same or different;
m is 1, 2, 3 or 4;
R2 is hydrogen or a substituent selected from the following: hydroxyl, amino, nitro, halogen, cyano,
wherein Y is absent or a group selected from:
R21, R22, R23, R24 are each independently hydrogen or a substituent selected from the group consisting of: C1-C6 alkyl, 3-8 membered cycloalkyl, 4-8 membered heterocycloalkyl, C2-C6 alkenyl, C2-C6 alkynyl, C1-C6 alkoxy; the C1-C6 alkyl, the 3-8 membered cycloalkyl, the 4-8 membered heterocycloalkyl, the C2-C6 alkenyl, the The C2-C6 alkynyl group or the C1-C6 alkoxy group is independently substituted by one or more Rf; when there are multiple substituents, the Rf is the same or different;
L is a group absent or selected from the group consisting of C1-C6 alkyl, deuterated C1-C6 alkyl, 3-8 membered cycloalkyl, 4-8 membered heterocycloalkyl, C2-C6 alkenyl, C2-C6 alkynyl, C1-C6 alkoxy; the C1-C6 alkyl, the 3-8 membered cycloalkyl, the 4-8 membered heterocycloalkyl, the C2-C6 alkenyl, the The C2-C6 alkynyl group or the C1-C6 alkoxy group is independently substituted by one or more Rf; when there are multiple substituents, the Rf is the same or different;
M is a substituent that is absent or selected from the group consisting of hydroxyl, amino, nitro, halogen, cyano, C1-C6 alkyl, C1-C6 alkoxy, —C00C1-C6 alkyl; the C1-C6 alkane group, the C1-C6 alkoxy group, or the —C00C1-C6 alkyl group are independently substituted by one or more Rf; when there are multiple substituents, the Rf is the same or different;
Ring A is absent or selected from 3-15-membered cycloalkyl or 4-15-membered heterocycloalkyl, 5-15-membered aryl or 5-15-membered heteroaryl;
Rb is independently hydrogen or a group selected from the group consisting of hydroxyl, amino, nitro, halogen, cyano, _NER5-C1-C6 alkyl, C1-C6 alkoxy, C2-C6 amido, C1-C6 ester group, C1-C6 carbonyl group; the C1-C6 alkyl group, the C1-C6 alkoxy group, the C2-C6 amido group, the C1-C6 ester group, or the C1-C6 carbonyl group independently be substituted by one or more Rf; when there are multiple substituents, the Rf is the same or different; n is 1, 2, 3 or 4; when there are multiple Rbs, Rb is the same or different substituents;
R3 is hydrogen or a substituent selected from the group consisting of halogen, cyano, C1-C6 alkyl, C1-C6 alkoxy, 3-8 membered cycloalkyl, 4-8 membered heterocycloalkyl;
the C1-C6 alkyl, the C1-C6 alkoxy, the 3-8 membered cycloalkyl, or the 4-8 membered heterocycloalkyl are each independently substituted with one or more Rf; when there are multiple substituents, the Rf is the same or different;
R4 is hydrogen or a substituent selected from the group consisting of halogen, C1-C6 alkyl, C1-C6 alkoxy, 3-8 membered cycloalkyl; the C1-C6 alkyl, the C1-C6 alkoxy, the 3-8 membered cycloalkyl is independently substituted by one or more Rf; when there are multiple substituents, the Rf is the same or different;
R5 is hydrogen or a substituent selected from the group consisting of halogen, C1-C6 alkyl, C1-C6 alkoxy; said C1-C6 alkyl or said C1-C6 alkoxy are independently replaced by one or more Rf is substituted; when there are multiple substituents, the Rf is the same or different;
R6 is a 5-8 membered cycloalkyl group, or a 5-8 membered aromatic ring group, or a 5-membered heteroaromatic ring group; the 5-8 membered cycloalkyl group, or the 5-8 membered aromatic ring group, Or a 5-10 membered heteroaryl ring is optionally substituted with one or more, the same or different substituents selected from the group consisting of: hydroxyl, amino, nitro, halogen, cyano, —SF5, C1-C6 alkyl, C2-C6 alkenyl, C2-C6 alkynyl, C1-C6 alkoxy, 3-8-membered cycloalkyl, 4-10-membered heterocycloalkyl, —SO2-C1-C6 alkyl; the hydroxyl, Amino is optionally substituted by C1-C6 alkyl, 3-8-membered cycloalkyl or 4-10-membered heterocycloalkyl; wherein, the C1-C6 alkyl, C2-C6 alkenyl, C2-C6 alkynyl, C1-C6 alkoxy, 3-8-membered cycloalkyl, 4-10-membered heterocycloalkyl are independently substituted by one or more Rf; when there are multiple substituents, the Rf is the same or different;
The Rf is a substituent selected from the group consisting of hydroxyl, amino, nitro, halogen, cyano, C1-C6 alkyl, C1-C6 haloalkyl, C1-C6 alkoxy, C1-C6 haloalkoxy, 3-8-membered cycloalkyl, 3-8-membered halogenated cycloalkyl, 4-10-membered heterocycloalkyl, C1-C6 acyl, C1-C6 carbonyl, C1-C6 sulfone, C1-C6 halogenated sulfone;

In the heterocycloalkyl or heteroaryl ring group, the heteroatom is selected from one or more of N, O, S and P, and the number of heteroatoms is 1-3.
(d) Wo2022/161461

A compound of formula (I) [symbol]
1. [symbol] (1)

[symbol]

[symbol]

[symbol]

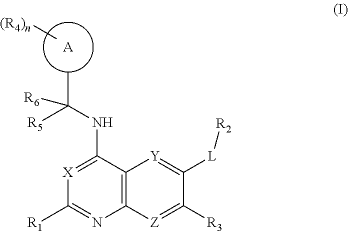

or a pharmaceutically acceptable salt thereof,
in,
X is selected from CH or N;
Y is selected from CH or N;
Z is selected from CH or N;
R1 is selected from H, CN, C1-6 alkyl or C3-6 cycloalkyl;
Ring A is selected from C6-10 aryl, benzo 5-7 membered heterocyclyl or benzo 5-7 membered heteroaryl;
L is selected from chemical bonds or O;
R2 is selected from C3-10 cycloalkyl, C6-10 aryl, 3-10-membered heterocyclyl or 5-10-membered heteroaryl, the C3-10 cycloalkyl, C6-10 aryl, 3-10 A membered heterocyclyl or 5-10 membered heteroaryl is optionally substituted with R2b and/or R2c;
R2b is selected from —OR2c, —N(R2c)2, halogen, hydroxyl, cyano, amino, —C(O)R2c, —C(O)NHR2c, —C(O)NH2, —NHR2c, —C(O)H, —C(O)OH, —S(O)2NHR2c, —NHC(O)H, —N(C1-4 alkyl)C(O)H, —C(O)N(R2c)2, —C(O)OR2c, —S(O)2R2c, —S(O)2N(R2c)2, —NHC(O)R2c or —N(C1-4 alkyl)C(O)R2c;
R2c is independently selected from C1-6 alkyl, C1-3 deuterated alkyl, C3-10 cycloalkyl, C6-10 aryl, 3-10 membered heterocyclyl or 5-10 membered heteroaryl, the C1-6 alkyl, C3-10 cycloalkyl, C6-10 aryl, 3-10 membered heterocyclyl, 5-10 membered heteroaryl optionally substituted by R2d;
R2d is selected from halogen, hydroxyl, cyano, amino, —C(O)R2f, —C(O)N(R2f)2, —C(O)OR2f, —S(O)2R2f, —S(O)2N(R2f)2, —N(C1-4 alkyl)R2f, —NHC(O)R2f or —N(C1-4 alkyl)C(O)R2f;
R2f is independently selected from H or C1-6 alkyl;
R3 is selected from H, halogen, hydroxyl, cyano, amino, —NH—C3-6 cycloalkyl, C1-3 deuterated alkyl, —O—C1-3 deuterated alkyl, C1-6 alkyl, C3-6 cycloalkyl, 3-8 membered heterocyclyl, —O—C1-6 alkyl, —O—C3-6 cycloalkyl, —O-(3-8 membered heterocyclyl), 5-10 membered Heteroaryl, —C(O)R3a, —C(O)N(R3a)2, —C(O)OR3a, —S(O)2R3a, —S(O)2N(R3a)2, —NHC(O)R3a or —N(C1-4 alkyl)C(O)R3a, the —NH—C3-6 cycloalkyl, C1-6 alkyl, C3-6 cycloalkyl, 3-8 membered heterocycle group, —O—C1-6 alkyl, —O—C3-6 cycloalkyl, —O-(3-8 membered heterocyclyl) or 5-10 membered heteroaryl optionally substituted by R3b;
The R3a is independently selected from H or C1-6 alkyl;
The R3b is independently selected from halogen, hydroxyl, cyano, amino, 3-8 membered heterocyclyl or C1-6 alkyl;
R4 is selected from halogen, hydroxyl, cyano, amino, C1-6 alkyl, C3-6 cycloalkyl, —O—C1-6 alkyl, —O—C3-6 cycloalkyl, —O-(3-8-membered heterocyclyl), 3-8 membered heterocyclyl, 5-10 membered heteroaryl or —S(O)2-C1-4 alkyl, the C1-6 alkyl, C3-6 cycloalkyl, —O—C1-6 alkyl, —O—C3-6 cycloalkyl, —O-(3-8 membered heterocyclyl), 3-8 membered heterocyclyl or 5-10 membered heteroaryl optional is substituted by R4a; said R4a is selected from halogen, hydroxy, cyano or amino;
R5 is selected from C1-3 deuterated alkyl, C1-6 alkyl or C1-6 haloalkyl;
R6 is selected from H, deuterium, C1-3 deuterated alkyl, C1-6 alkyl or C1-6 haloalkyl;
n is selected from 0, 1, 2, 3 or 4;
Wherein, when X is selected from N, Z is selected from CH, R is selected from CN or C3-6 cycloalkyl;
When X and Z are selected from N, R1 is selected from CN, C1-6 alkyl or C3-6 cycloalkyl;
When X and Z are selected from CH, R3 is selected from hydroxyl, cyano, amino, C1-3 deuterated alkyl, —O—C1-3 deuterated alkyl, C1-6 alkyl, C3-6 cycloalkyl, 3-8 membered heterocyclyl, —O—C1-6 alkyl, —O—C3-6 cycloalkyl, —O-(3-8 membered heterocyclyl), 5-10 membered heteroaryl, —C(O)R3a, —C(O)N(R3a)2, —C(O)OR3a, —S(O)2R3a, —S(O)2N(R3a)2, —NHC(O)R3a or —N(C1-4 alkyl)C(O)R3a, the C1-6 alkyl, C3-6 cycloalkyl, 3-8 membered heterocyclyl, —O—C1-6 alkyl, —O—C3-6 cycloalkyl, —O-(3-8 membered heterocyclyl) or 5-10 membered heteroaryl optionally substituted with R3b.
   (e) Wo2022/166974

Pyridopyrimidone derivatives represented by formula I,

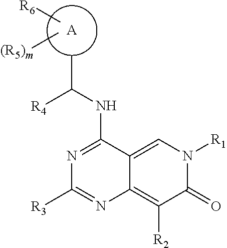

their tautomers, stereoisomers, hydrates, solvates, pharmaceutically acceptable salts or prodrugs:
Wherein, ring A is a 6-10-membered aromatic ring or a 9-11-membered heteroaromatic ring;
R1 is a 3-10 membered cycloalkyl or a 4-10 membered heterocycloalkyl, the R1 is optionally substituted with one or more R11, and the R11 is a substituent selected from the group consisting of halogen, hydroxyl, C1-C6 alkyl, C1-C6 haloalkyl, C1-C6 alkoxy, When there are multiple substituents R11, the substituents R11 are the same or different;
The R11 is optionally substituted with a substituent selected from the group consisting of C1-C6 alkyl, C1-C6 alkoxy, halogen, hydroxyl; R12 is C1-C6 alkyl, one or more F-substituted C1-C6 Alkyl or 3-6 membered cycloalkyl;
R13 is hydrogen, C1-C6 alkyl or cyano;
R14 is hydrogen, C1-C6 alkyl, C1-C6 haloalkyl;
R2 is hydrogen or a substituent selected from the following: halogen, C1-C6 alkyl, 3-6 membered cycloalkyl, C1-C6 alkoxy; the C1-C6 alkyl, 3-6 membered cycloalkyl, C1-C6 alkoxy is independently substituted by one or more R21; the R21 is a substituent selected from the following: hydroxyl, halogen, C1-C3 alkoxy; when there are multiple substituents, the R21 same or different;
R3 is hydrogen or a substituent selected from the group consisting of halogen, C1-C6 alkyl, C1-C6 haloalkyl;
R4 is C1-C6 alkyl or C1-C6 haloalkyl;
R5 is hydrogen or a substituent selected from the group consisting of halogen, C1-C6 alkyl, C1-C6 haloalkyl;
R6 is —SF5, described R61, described R62 are each independently halogen-substituted C1-C6 alkyl or 3-6 membered cycloalkyl;
Or ring A and R6, R5 together form group fragment
wherein, Z is R63 is hydrogen or a substituent selected from the following: halogen, hydroxyl, C1-C6 alkyl, halogen substituted C1-C6 alkyl; When the substituent R63 is multiple, the R63 is the same or different;
m is 1 or 2; p is 1, 2 or 3; n is 1, 2 or 3.
   (f) Wo2022/170917

A polycyclic pyrimidine derivative, its pharmaceutically acceptable salt, its tautomer or its stereoisomer, characterized in that the structure of said polycyclic pyrimidine derivative is as shown in formula (I)

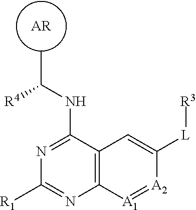

Wherein: R1 is selected from hydrogen or C1-C3 alkyl; preferably hydrogen or methyl;
A1 is selected from N or C—R11;
R11 is selected from H, C1-C3 alkyl or C1-C3 haloalkyl;
A2 is selected from N or C—R2;
R2 is selected from —OR21 or cyano;
R21 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R22;
R22 is selected from halogen, C1-C4 alkyl, cyano, hydroxyl;
L is absent or selected from O, NH or NCH3;
R3 is selected from 3-7 membered cycloalkyl, 4-7 membered heterocyclyl, 6-10 membered fused heterocyclyl, 6-10 membered bridged heterocyclyl, 6-10 membered spiro heterocyclyl, wherein 3-7 membered cycloalkyl, 4-7 membered heterocyclyl, 6-10 membered fused heterocyclyl, 6-10 membered bridged heterocyclyl, 6-10 membered spiro heterocyclyl optionally—3 R31 replaced;
R31 is selected from C1-C3 alkyl, C1-C3 haloalkyl, hydroxy, halogen, cyano, —NRaRb, C1-C3 alkoxy, ═O, —NHCOR32 or —COR32;
R32 is selected from C1-C3 alkyl, C1-C3 haloalkyl, 3-7 membered cycloalkyl or 4-7 membered heterocyclyl;
R4 is —CH3;
AR is selected from 6-10-membered aryl or 5-10-membered heteroaryl, wherein the aryl or heteroaryl is optionally substituted by 1-4 R5;
R5 is selected from halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —ORa, —NRaRb;
Ra is selected from H, C1-C4 alkyl, C1-C4 haloalkyl, 3-6 membered cycloalkyl or 4-7 membered heterocyclyl;
Rb is selected from H, C1-C4 alkyl, C1-C4 haloalkyl, 3-6 membered cycloalkyl or 4-7 membered heterocyclyl;

In the above definition, the following conditions cannot occur at the same time:
1) A1 is C—R11;
2) A2 is C—OR21;
3) L is 0 or does not exist;

The heteroatoms in the heterocyclic group and the heteroaryl group in the formula (I) are 1-3 and are selected from one or more of oxygen, nitrogen and sulfur.
(g) Wo2022/171018

Substituted benzo or pyridopyrimidine amine compounds with general formula (I), their stereoisomers, tautomers, crystal forms, pharmaceutically acceptable salts, hydrates, solvates or prodrugs:

In the formula, [symbol]
1 [symbol] (I) [symbol] [symbol] [symbol] [symbol] [symbol] [symbol] [symbol] [symbol] [symbol] [symbol] :

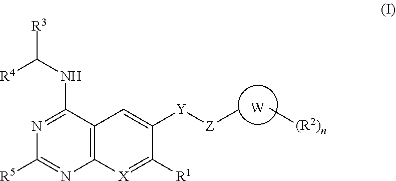

heterocyclyl;
Z is selected from the group consisting of substituted or unsubstituted: bond, substituted or unsubstituted C1-C18 alkylene;
W is selected from the group of substituted or unsubstituted groups: bond, C3-C20 cycloalkylene, 4-20 membered heterocyclylene, OR11, NR11R12, SO2, NR12SO2, CO or NR12CO; R11 is independently selected from substituted or unsubstituted following groups: C3-C20 cycloalkylene, 4-20-membered heterocyclylene, C3-C20 cycloalkylene C1-C18 alkylene, 4-20-membered heterocyclylene C1-C18 alkylene, C6-C14 aryl or 5-14 membered heteroaryl; R12 is independently selected from the group consisting of substituted or unsubstituted hydrogen, deuterium, C1-C6 alkyl or C3-C6 cycloalkyl;
R1 and R2 are each independently selected from the group consisting of hydrogen, deuterium, halogen, cyano, —(CH2)mR8, —(CH2)m(CH═CH)R8, —(CH2)m(C≡C)R8, —(CH2)mO(CH2)pR8, —(CH2)mSR8, —(CH2)mCOR8, —(CH2)mC(O)OR8, —(CH2)mS(O)qR8, —(CH2)mNR8R9, —(CH2)mC(O)NR8R9, —(CH2)mNR8C(O)R9, —(CH2)mNR8C(O)NR9R10, —(CH2)mS(O)qNR8R9, —(CH2)mNR8S(O)qR9, —(CH2)mNR8S(O)qNR9R10, wherein, H in CH2 can be optionally substituted; R8, R9, R10 are each independently selected from the group of substituted or unsubstituted groups: hydrogen, C1-C18 alkyl, C1-C18 alkoxy, C3-C20 cycloalkyl, 4-20 membered heterocyclyl, C6-C14 aryl or 5-14 membered heteroaryl; or in —(CH2)mNR8R9, —(CH2)mC(O) NR8R9, —(CH2)mS(O)qNR8R9, R8 and R9 are cyclized with their adjacent N atoms to form a substituted or unsubstituted 4-8-membered heterocyclic group; or in —(CH2)mNR8C(O)R9, —(CH2)mNR8C(O)NR9R10, —(CH2)mNR8S(O)qR9, —(CH2)mNR8S(O)qNR9R10, R8 and R9 are cyclized with their adjacent N atoms to form substituted or unsubstituted 4-8-membered heterocyclyl, or R9 and R10 are cyclized with their adjacent atoms to form a substituted or unsubstituted 4-8-membered heterocyclyl;
R3 is selected from the group consisting of substituted or unsubstituted groups: C3-C18 cycloalkyl, 4-20-membered heterocyclyl, C6-C14 aryl, 5-14-membered heteroaryl;
R4 and R5 are each independently selected from the group consisting of substituted or unsubstituted groups: C1-C6 alkyl, C3-C6 cycloalkyl, 4-6 membered heterocyclic group, ester group, COOH, CONH2, C2-C6 alkene base, C2-C6 alkynyl;
Wherein, the above-mentioned substitution refers to being substituted by one or more groups selected from the group consisting of hydrogen, deuterium, C1-C18 alkyl, deuterated C1-C18 alkyl, halogenated C1-C18 alkyl, halogenated C1-C18 alkylhydroxy, C3-C20 cycloalkyl, C3-C20 cycloalkyl-O—, C1-C18 alkoxy, deuterated C1-C18 alkoxy, halogenated C1-C18 alkoxy, C6-C14 Aryl, 5-14-membered heteroaryl, 4-20-membered heterocyclyl, 4-20-membered heterocyclyl-O—, halogen, oxo C1-C6 alkyl, nitro, hydroxyl, cyano, C2-C6 ester group, C1-C6 amine group, C2-C6 acyl group, C1-C6 amide group, C1-C6 sulfonyl group, C1-C6 sulfonamido group or C1-C6 urea group; wherein, the C1-C18 alkyl group, Deuterated C1-C18 alkyl, halogenated C1-C18 alkyl, halogenated C1-C18 alkyl hydroxyl, C3-C20 cycloalkyl, C3-C20 cycloalkyl-O—, C1-C18 alkoxy, deuterium Substituted C1-C18 alkoxy, halogenated C1-C18 alkoxy, C6-C14 aryl, 5-14-membered heteroaryl, 4-20-membered heterocyclyl, 4-20-membered heterocyclyl-O— also Can be further substituted by one or more Ra, wherein, Ra is selected from: C1-C6 alkyl, deuterated C1-C6 alkyl, halogenated C1-C6 alkyl, halogenated C1-C6 alkyl hydroxyl, C3-C6 Cycloalkyl, C3-C6 cycloalkyl-O—, C1-C6 alkoxy, deuterated C1-C6 alkoxy, halogenated C1-C6 alkoxy, C6-C14 aryl, 5-14 membered hetero Aryl, 4-6 membered heterocyclyl, 4-6 membered heterocyclyl-O—, halogen, oxo C1-C6 alkyl, nitro, hydroxyl, cyano, C2-C6 ester, C1-C6 amine group, C2-C6 amide group, C1-C6 sulfonamido group or C1-C6 urea group; or two substituents located on the same carbon atom together form —(CH2)n— or ═O;
m, n are each independently 0, 1, 2, 3, 4 or 5;
p is 0, 1, 2, 3, 4, or 5;
q is 1 or 2;

The limitation is that when Y is selected from the following group: O, NH or NR7, and Z is a bond, W is a C3-C20 cycloalkylene group or a 4-20-membered heterocyclic group; R1 is not hydrogen, deuterium, halogen, Cyano, R8, O(CH2)pR8, COR8, —C(O)OR8, NR8R9, C(O)NR8R9, —NR8C(O)R9, —NR8C(O)NR9R10.
(h) WO2022/170802

A pyrimidopyridone derivative, a pharmaceutically acceptable salt thereof, a tautomer or a stereoisomer thereof, wherein the structure of the pyrimidopyridone derivative is as shown in formula (I) as shown:

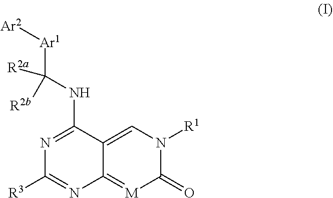

Wherein: R1 is selected from hydrogen or C1-C3 alkyl;
R2 is selected from hydrogen or C1-C3 alkyl, 3-7 membered cycloalkyl, 4-7 membered heterocyclyl, wherein C1-C3 alkyl, 3-7 membered cycloalkyl, 4-7 membered heterocyclyl are any optionally replaced by 1-3 R21;
R21 is selected from C1-C3 alkyl, hydroxyl, halogen, cyano, amino, C1-C3 alkoxy or ═o;
L may be absent or selected from O, NH or N—(C1-C3 alkyl);
R3 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R31;
R31 is selected from C1-C3 alkyl, C1-C3 haloalkyl, hydroxy, halogen, cyano, —NRaRb, C1-C3 alkoxy, ═O, —NHCOR32 or —COR32;
Ra is selected from H, C1-C3 alkyl, C1-C3 haloalkyl or 3-6 membered cycloalkyl;
Rb is selected from H, C1-C3 alkyl, C1-C3 haloalkyl or 3-6 membered cycloalkyl;
R32 is selected from C1-C3 alkyl, C1-C3 haloalkyl, 3-6 membered cycloalkyl or 4-7 membered heterocyclyl;
AR is selected from 6-10-membered aryl or 5-10-membered heteroaryl, wherein the aryl or heteroaryl is optionally substituted by 1-4 R4;
R4 is selected from H, halogen, C1-C3 alkyl, C1-C3 haloalkyl, hydroxy-C1-C3 alkyl, hydroxy-C1-C3 haloalkyl, 3-6 membered cycloalkyl, 4-7 membered heterocyclyl, —ORa, —NRaRb, 6-10-membered aryl or 5-10-membered heteroaryl, wherein the 6-10-membered aryl or 5-10-membered heteroaryl is optionally replaced by 1-4 Rc replaced;
Rc is selected from H, halogen, C1-C3 alkyl, C1-C3 haloalkyl, hydroxy-C1-C3 alkyl, hydroxy-C1-C3 haloalkyl, 3-6 membered cycloalkyl, 4-7 membered heterocyclyl, —ORa, —NRaRb, NRaRb-C1-C4 alkyl, NRaRb-C1-C4 haloalkyl; the heteroatoms in the heterocyclic group or heteroaryl in the formula (I) are 1-3 and are selected from One or more of oxygen, nitrogen and sulfur.
(i) WO2022/170952

A polycyclic pyridazinone derivative, a pharmaceutically acceptable salt thereof, a tautomer or a stereoisomer thereof,
It is characterized in that, the structure of the polycyclic pyridazinone derivatives is shown in formula (I):

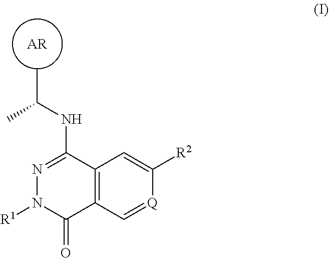

Wherein: R1 is selected from hydrogen or methyl;
R2 is selected from C1-C3 alkyl, —OR21, halogen, 3-7 membered cycloalkyl, 5-7 membered cycloalkenyl, 6-10 membered fused cycloalkyl, 7-10 membered bridged cycloalkyl, 7-10 membered spirocycloalkyl, 4-7 membered heterocyclyl, 5-7 membered heterocycloalkenyl, 6-10 membered fused heterocyclyl, 7-10 membered bridged heterocyclyl, 7-10 membered heterocyclyl A membered spiro heterocyclic group, wherein 3-7 membered cycloalkyl, 5-7 membered cycloalkenyl, 6-10 membered fused cycloalkyl, 7-10 membered bridged cycloalkyl, 7-10 membered spiro Cycloalkyl, 4-7 membered heterocyclyl, 5-7 membered heterocyclenyl, 6-10 membered fused heterocyclyl, 7-10 membered bridged heterocyclyl, 7-10 membered spiro heterocyclyl optionally substituted by 1-3 R22;
R21 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R22;
R22 is selected from C1-C3 alkyl, hydroxyl, halogen, cyano, —NRaRb, C1-C3 alkoxy, —C(O)Ra, —C(O)ORa, —OC(O)Ra, —NRbC(O)Ra, —NRbC(O)ORa, —C(O)NRaRb, phenyl, 5-6 membered heteroaryl and ═O, wherein alkyl, alkoxy, phenyl, 5-6 membered heteroaryl group is optionally further substituted with 1-3 halogen, C1-C3 alkyl, hydroxy, cyano, amino and C1-C3 alkoxy;
Ra and Rb are independently selected from H, substituted or unsubstituted C1-C3 alkyl, substituted or unsubstituted 3-6 membered cycloalkyl, or substituted or unsubstituted 4-7 membered heterocyclyl; here “Substituted” means optionally substituted with 1-3 substituents selected from C1-C3 alkyl, hydroxy, halogen, cyano, amino or alkoxy;
Q is selected from N or —CR3;
R3 is selected from H, C1-C3 alkyl, halogen, cyano or —OR21;
AR is selected from 6-10-membered aryl or 5-10-membered heteroaryl, wherein the aryl or heteroaryl is optionally substituted by 1-4 Rc;
Rc is selected from H, halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —OR21, —NRaRb, NRaRb-C1-C4 alkyl, NRaRb-C1-C4 haloalkyl, 6-10-membered aryl or 5-10-membered heteroaryl, wherein 6-10-membered aryl or 5-10 membered heteroaryl is optionally substituted with 1-4 Rd;
Rd is selected from H, halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —OR21, —NRaRb, NRaRb-C1-C4 alkyl, NRaRb-C1-C4 haloalkyl;

The heteroatoms in the heterocyclic group, heteroaryl group, heterocyclic alkenyl group, condensed heterocyclic group, bridged heterocyclic group and spiro heterocyclic group in the formula (I) are 1-7 and are selected from oxygen, nitrogen One or more of, sulfur and S(O)m, where m is 1 or 2.
(j) WO2022/187236

A compound of Formula (I) or Formula (II),

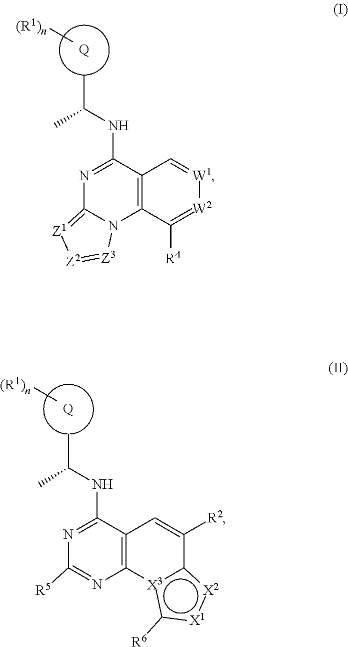

or a pharmaceutically acceptable salt thereof, and/or a tautomer thereof, and/or a stereoisomer thereof,
wherein:
Q at each occurrence is independently a ring selected from phenyl or a 5- or 6-membered heteroaryl group, wherein the heteroaryl group comprises at least one carbon atom and 1-4 additional heteroatoms independently selected from the group consisting of nitrogen, oxygen and sulfur;
Z¹ is N or CR5; Z² is N or CR⁶;
Z³ is N or CR⁷;
W¹ is CR² or NR²;
W² is CR³ when W¹ is CR², and W² is C(O) when
W¹ is NR²; X¹ is N, NR⁷, or CR⁹;
X² is N or CR⁷;
X³ is N or C;
R1 at each occurrence is independently hydrogen, halogen, C1-6 alkyl, haloC1-6 alkyl, C1-6 alkenyl, C1.5 alkynyl, —NRaRt, OH, C1-6 alkyl-OH, haloC1-6 alkyl-OH, C1-6 alkoxy, haloC1.5 alkoxy, CN, C3.1 cycloalkyl, C3.7 cycloalkyl-OH, C3.7 cycloalkoxy, —S(O)t-C1-oalkyl, —S(O)t-NRaR\ phenyl, or 3-7-membered heterocyclyl, wherein the phenyl and 3-7-membered heterocyclyl are optionally substituted with 1-4 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —S(O)t-C1-6 alkyl, —S(O)t-NRaRb, —NRaRb, or C1.4 alkyl-NRaRt; or two adjacent R¹ groups, together with the carbon atoms to which they are attached, form a 5-7-membered carbocyclic or heterocyclic ring optionally substituted with 1-3 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —NR3Rb, C1.4 alkyl-NRaRb, or oxo group (═O);
R2 at each occurrence is independently hydrogen, halogen, CN, —ORa, —NRaRh, C1-6 alkyl, haloC1-6 alkyl, C2-6 alkenyl, C2-6 alkynyl, C3.1 cycloalkyl, 3-7-membered heterocylyl, phenyl, or 5-6-membered heteroaryl, wherein each of the C1-6 alkyl, haloC1-6 alkyl, C2-6 alkenyl, C2-6 alkynyl, C3-7 Cycloalkyl, 3-7-membered heterocylyl, phenyl, and 5-6-membered heteroaryl is optionally substituted with 1-5 R⁸;
R³ at each occurrence is independently hydrogen, halogen, C1-6 alkyl, haloC1-6 alkyl, C1-6 alkoxy, haloC1-6 alkoxy, C1-6 alkyl-OH, CN, C3-1 cycloalkyl, C3.7 cycloalkyl-OH, C3.1 cycloalkoxy,
—NH2, —NHC1.4 alkyl, —N(C1.4 alkyl)2, or 3-7-membered cyclic amine;
R⁴ at each occurrence is independently hydrogen, halogen, C1-6 alkyl, haloC1-6 alkyl, C1-6 alkoxy, haloC1-nalkoxy, CN, NH2, C3-7 cycloalkyl or C1-1 cycloalkoxy;
R⁵ at each occurrence is independently hydrogen, C1.4 alkyl, or haloC1.4 alkyl;
R⁶ at each occurrence is independently hydrogen or C1.4 alkyl;
R⁷ at each occurrence is independently hydrogen or C1.4 alkyl;
R⁸ at each occurrence is independently hydrogen, halogen, C1.4 alkyl, haloC1.4 alkyl, C1.4 alkoxy, C2.4 alkenyl, C2.4 alkynyl, C3-7 cycloalkyl, C3-7 cycloalkoxy, 3-7-membered heterocyclyl, phenyl, 5-6-membered heteroaryl, —ORa, —SRa, S(O)tRa, —S(O)t-NRaR\—OC(O)—Ra, —NRaRb,
—C(O)Ra, —C(O)ORa, —OC(O)NRaRb2, —C(O)NRaRb, —N(Ra)C(O)ORa, —N(Ra)C(O)Ra, —N(Ra)C(O)NRaR\—N(Ra)C(NRa)NRaRb, —N(Ra)S(O)tNRaRb, —P(═O)(Ra)(Rb), —O—
P(═O)(ORa)(ORb), or oxo group (═O);
R⁹ at each occurrence is independently hydrogen or C1.4 alkyl;
Ra and Rb at each occurrence are independently hydrogen, C1-Galkyl, haloC1-Galkyl, C1-Galkyl-OH, C1-6 alkoxy, C3.1 cycloalkyl, 3-7-membered heterocyclyl, C1-6 alkyl-NH2, C1-6 alkyl-NHC1.4 alkyl, C1-6 alkyl-N(C1.4 alkyl)2, or C1-6 alkyl-(3-7-membered cyclic amine); or Ra and Rh, together with the nitrogen atom to which they are attached, form a saturated or unsaturated heterocyclic ring containing from three to seven ring atoms, which ring may optionally contain an additional one or two heteroatoms independently selected from the group consisting of nitrogen, oxygen and sulfur and which ring may be optionally substituted by from one to three substituents independently selected from the group consisting of C1-4 alkyl, phenyl and benzyl;
n at each occurrence is independently 1, 2 or 3, and that each occurrence is independently 1 or 2.
   (k) WO2022/187266
1. A compound of Formulae (I)-(IV),

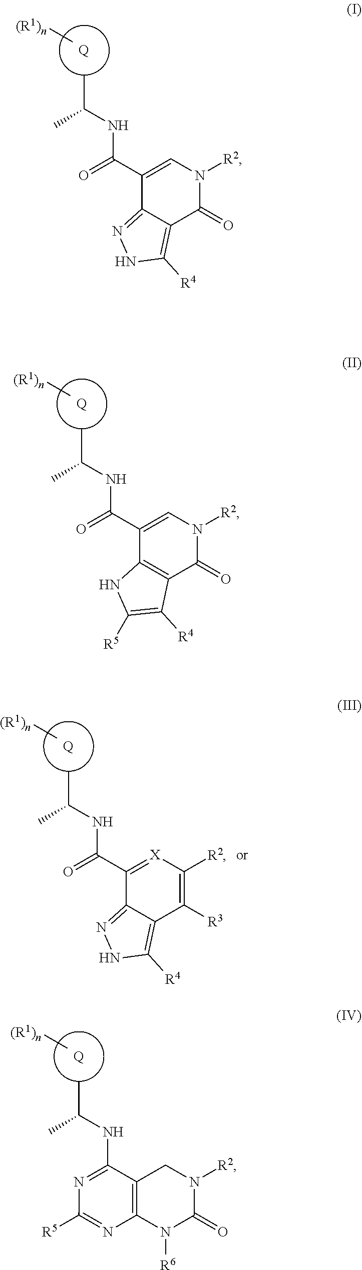

or a pharmaceutically acceptable salt, tautomer or stereoisomer thereof, wherein:
Q at each occurrence is independently a ring selected from phenyl or a 5- or 6-membered heteroaryl group, wherein the heteroaryl group comprises at least one carbon atom and 1-4 additional heteroatoms independently selected from nitrogen, oxygen and sulfur;
X is CH or N;
R¹ at each occurrence is independently hydrogen, halogen, C1-6 alkyl, haloC1-6 alkyl, C1-6 alkenyl, C1-6 alkynyl, —NR³Rb, OH, C1-6 alkyl-OH, haloC1-6 alkyl-OH, C1-6 alkoxy, haloC1-6 alkoxy, CN, C3-1 cycloalkyl, C3-7 cycloalkyl-OH, C3-7 Cycloalkoxy, —S(O)t-C1-6 alkyl, —S(O)t-NR³R\ phenyl, or 3-7-membered heterocyclyl, wherein the phenyl and 3-7-membered heterocyclyl are optionally substituted with 1-4 substituents independently selected from C1-4 alkyl, haloC1-4 alkyl, C1-4 alkoxy, haloC1-4 alkoxy, C1-4 alkyl-OH, haloC1-4 alkyl-OH, OH, halogen, CN, —S(O)t-C1-6 alkyl, —S(O)t-NRaRh, —NR³Rh, and C1-4 alkyl-NRaRh, or two adjacent R1 groups, together with the carbon atoms to which they are attached, form a 5-7-membered carbocyclic or heterocyclic ring optionally substituted with 1-3 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —NRaRh, C1.
4 alkyl-NR³Rh, and oxo group (═O);
R² at each occurrence is independently hydrogen, halogen, CN, —OR³, —NR³Rh, C1-6 alkyl, haloC1.6 alkyl, C2-6 alkenyl, C2-6 alkynyl, C3.7 cycloalkyl, 3-7-membered heterocylyl, phenyl, or 5-6-membered heteroaryl, wherein each of the C1-6 alkyl, haloC1-6 alkyl, C2-6 alkenyl, C2-6 alkynyl, C3.7 cycloalkyl, 3-7-membered heterocylyl, phenyl, and 5-6-membered heteroaryl is optionally substituted with 1-5 R⁸;
R³ at each occurrence is independently hydrogen, halogen, C1-6 alkyl, haloC1-6 alkyl, C1-6 alkoxy, haloC1-Galkoxy, C1-Galkyl-OH, CN, C3.1 cycloalkyl, C3.1 cycloalkyl-OH, C3.1 cycloalkoyx, —NH2, —NHC1.4 alkyl, —N(C1.4 alkyl)2, or 3-7-membered cyclic amine;
R⁴ at each occurrence is independently hydrogen, halogen, C1-6 alkyl, haloC1-6 alkyl, C1-6 alkoxy, haloC1-6 alkoxy, CN, NH2, C3.7 cycloalkyl or C3.1 cycloalkoxy;
R⁵ at each occurrence is independently hydrogen, C1.4 alkyl, or haloC1.4 alkyl;
R⁶ at each occurrence is independently hydrogen, C1-oalkyl, haloC1-oalkyl, or C1-1 cycloalkyl;
R⁸ at each occurrence is independently hydrogen, halogen, C1.4 alkyl, haloC1.4 alkyl, C1.4 alkoxy, C2.4 alkenyl, C2.4 alkynyl, C3.7 cycloalkyl, C3.7 cycloalkoxy, 3-7-membered heterocyclyl, phenyl, 5-6-membered heteroaryl, —OR³, —SR³, S(O)tR³, —S(O)tNR³Rh, —OC(O)—R³, —NR³Rh, —C(O)R³, —C(O)OR³, —OC(O)NR³Rb2, —C(O)NR³Rb, —N(R³)C(O)OR³, —N(R³)C(O)R³,
N(R³)C(O)NR³R\—N(R³)C(NR³)NR³R\—N(R³)S(O)tNR³R\—P(═O)(R³)(Rb), —O—P(═O)(OR³)(ORb), or oxo group (═O);
R³ and Rb at each occurrence are independently hydrogen, C1-6 alkyl, haloC1-6 alkyl, C1-6 alkyl-OH, C1-6 alkoxy, C3-7 cycloalkyl, 3-7-membered heterocyclyl, C1-6 alkyl-NH2, C1-6 alkyl-NHC1.4 alkyl, C1-6 alkyl-N(C1.4 alkyl)2, or C1-6 alkyl-(3-7-membered cyclic amine), wherein each of the foregoing groups may be optionally substituted by one to three substituents independently selected from the group consisting of C1-4 alkyl, haloC1.4 alkyl, halogen, OH, NH2, C1.4 alkoxy, haloC1.4 alkoxy, CN, and —C(O)C1.4 alkyl; or Ra and Rh, together with the nitrogen atom to which they are attached, form a saturated or unsaturated heterocyclic ring containing from three to seven ring atoms, which ring may optionally contain an additional one or two heteroatoms independently selected from the group consisting of nitrogen, oxygen and sulfur and may be optionally substituted by from one to three substituents independently selected from the group consisting of C1-4 alkyl, —C(O)C1.4 alkyl, phenyl and benzyl;
n at each occurrence is independently 1, 2 or 3, and that each occurrence is independently 1 or 2.
   (l) WO2022/146698

A compound having a structure of Formula (I), Formula (II), or Formula (III):

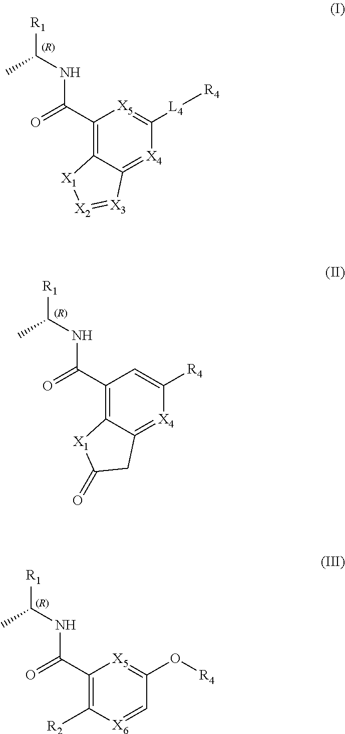

or a pharmaceutically acceptable salt, solvate, stereoisomer, prodrug, or tautomer thereof, wherein:
X1 is NH or S;
X2 is CH or N;
X3 is CH or N;
X4 is CR³ or N
X5 is CH or N;
X6 is CH or N;
R1 is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R2 is selected from the group consisting of H, —NH—C1-6 alkyl, and —NH2;
R3 is selected from the group consisting of H, —O—C1-6 alkyl, and —O—C1-6 heteroalkyl;
L4 is a bond or O; and
   R4 is selected from the group consisting of H, C1-6 alkyl, 3-14 membered cycloalkyl, 3-14 membered cycloalkenyl, 3-14 membered heterocyclyl, 6-10 membered aryl, and 5-10 membered heteroaryl; wherein each C1-6 alkyl, 3-14 membered cycloalkyl, 3-14 membered cycloalkenyl, 3-14 membered heterocyclyl, 6-10 membered aryl, and 5-10 membered heteroaryl is optionally substituted with one or more C1-6 alkyl, -R4a, —OR4a, —O—C1-6 alkyl-R4a, ═O, halogen, —C(O)R4a, —C(O)OR4a, —C(O)NR4bR4c, —NR4bC(O)R4c, —CN, ═NR4a, —NR4bR4c, —SO2R4a, 3-6 membered cycloalkyl optionally substituted with R4a, 3-7 membered heterocyclyl optionally substituted with R4a, 6-10 membered aryl optionally substituted with R4a, or 5-10 membered heteroaryl optionally substituted with R4a;
   wherein R4a is H, C1-6 alkyl, C1-6 haloalkyl, —C(O)R4b, —C(O)NR4bR4c, ═O, 3-6 membered cycloalkyl, 6-10 membered aryl optionally substituted with —OR4b, —CN, ═N-3-6 membered cycloalkyl, 3-7 membered heterocyclyl, —(CH2)rOCH3, or —(CH2)rOH, wherein r is 1, 2, or 3;
      wherein each R4b is independently H, C1-6 alkyl; and
      wherein each R4c is independently H or C1-6 alkyl.
(m) WO2022/140427

A compound of formula I:

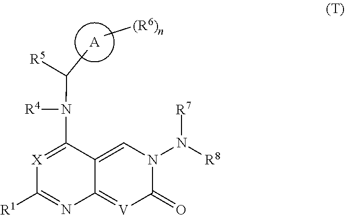

a pharmaceutically acceptable salt or a stereoisomer thereof, wherein:
   ring A is 3-12 membered carbocyclyl, 3-12 membered heterocyclyl, 6-10 membered aryl, or 5-10 membered heteroaryl;
   R¹ is hydrogen, halogen, C1-6 alkyl, or C3-6 cycloalkyl, wherein said C1-6 alkyl or
C3-6 cycloalkyl represented by R¹ is optionally substituted by one to more groups selected from halogen and —OH;
   V is N or CR²; wherein
   R² is hydrogen, halogen, —CN, C1-6 alkyl, C2-6 alkenyl, C2-6 alkynyl, —OR²U, —NR²aR²h,
—C(O)R²U, —C(O)OR²a, —C(O)NR²aR²h, —SO2R²U, —SO2NR²aR²h, —P(O)R²aR²h, —NR² aC(O)R²h,
—NR² aC(O)OR²h, —NR² aSO2R²h, —NR² aSO2NR²bR2c, 3-12 membered carbocyclyl,
3-12 membered heterocyclyl, 6-10 membered aryl, or 5-10 membered heteroaryl; wherein
said C1-6 alkyl, C2-6 alkenyl, C2-6 alkynyl, 3-12 membered carbocyclyl, 3-12 membered heterocyclyl, 6-10 membered aryl, or 5-10 membered heteroaryl represented by R² is optionally substituted by one or more R²d; wherein
   R²a, R²b, and R²c are independently selected from the group consisting of hydrogen, C1-6 alkyl, 3-12 membered carbocyclyl, 3-12 membered heterocyclyl, 6-10 membered aryl, and 5-10 membered heteroaryl; or R² and R²h or R²h and R²c together with the N or P atom to which they are attached form 4-12 membered heterocyclyl or 5-10 membered heteroaryl; wherein said C1-6 alkyl, carbocyclyl,
heterocyclyl, aryl, or heteroaryl represented by R²U, R²b, or R²c or in the group represented by R²U, R²h, or R²c are optionally substituted with one or more R²d; wherein
   R²d, in each occurrence, is hydrogen, halogen, oxo, —CN, C1-6 alkyl, C1-6haloalkyl, —OR²\—NR²eR²r, —C(O)R²\—C(O)OR²\—C(O)NR²eR²r, —SO2R²\—SO2NR²eR²r, —P(O)R²eR²r, —NR²eC(O)R²r, —NR²eC(O)OR²r, —NR²eSO2R²r, —NR²eSO2NR²rR²g, 3-12 membered carbocyclyl, 3-12 membered heterocyclyl, 6-10 membered aryl, or 5-10 membered heteroaryl;
      R²e, R²r, and R²g are independently selected from the group consisting of hydrogen and C1-6 alkyl;
   X is N or CR³;
   R³ is hydrogen, halogen, or C1-3 alkyl;
   R⁴ is hydrogen or C1-6 alkyl;
   R⁵ is hydrogen, C1-6 alkyl, 3-6 membered monocyclic carbocyclyl, or 4-6 membered monocyclic heterocyclyl; wherein said C1-6 alkyl, 3-5 membered monocyclic carbocyclyl, or 4-6 membered monocyclic heterocyclyl represented by R⁵ is optionally substituted with one or more groups selected from halogen and —OH;
   R⁶ is hydrogen, —OH, halogen, —CN, oxo, C1-6 alkyl, C1-6 alkoxy, —SO2R⁶\—SO2NR⁶aR⁶b, —P(O)R⁶aR⁶b, —C(O)NR⁶aR⁶b, —NR⁶ aC(O)R⁶—NR⁶ aC(O)NR⁶aR⁶b, —(CH2)sNR⁶aR⁶h, —O(CH2)rNR⁶aR⁶h, 3-12 membered carbocyclyl, 3-12 membered heterocyclyl, 6-10 membered aryl, 5-membered heteroaryl; wherein said C1-6 alkyl, C1-6 alkoxy, 3-12 membered carbocyclyl, 3-12 membered heterocyclyl, 6-membered aryl, or 5-10 membered heteroaryl represented by R⁶ is optionally substituted by one to more R⁶c; wherein R⁶a and R⁶b are independently hydrogen or C1-6 alkyl, or R⁶a and R⁶b together with the N or P atom to which they are attached form 4-7 membered heterocyclyl;
      s is an integral from 0 to 3;
      t is an integral from 2 to 4;
      R⁶C, in each occurrence, is hydrogen, —OH, halogen, —CN, oxo, C1-6 alkyl, C1-6 alkoxy, C3-6 cycloalkyl, —NR⁶aR⁶—SO2R⁶—SO2NR⁶aR⁶h, —C(O)NR⁶aR⁶h, —P(O)R⁶aR⁶b, —NR⁶ aC(O)R⁶U, —NR⁶ aC(O)NR⁶aR⁶b, —(CH2), NR⁶aR⁶b, or —O(CH2)rNR⁶aR⁶b; wherein said C1-6 alkyl or C3-6 Cycloalkyl represented by R⁶c is optionally substituted with one to more groups selected from halogen, —OH and —NR⁶aR⁶b;
   R⁷ and R⁸ are independently hydrogen, C1-6 alkyl, C3-6 alkenyl, C3-6 alkynyl, C2-6 alkoxy, 3-12 membered carbocyclyl, 3-12 membered heterocyclyl, 6-10 membered aryl, or 5-10 membered heteroaryl; wherein said C1-6 alkyl, C3-6 alkenyl, C3-6 alkynyl, C2-6 alkoxy, 3-12 membered carbocyclyl, 3-12 membered heterocyclyl, 6-10 membered aryl, or 5-10 membered heteroaryl represented by R⁷ or R⁸ is optionally substituted by one or more R⁷a; or
   R⁷ and R⁸ together with the N atom to which they are attached form 4-12 membered heterocyclyl or 5-10 membered heteroaryl; wherein said 4-12 membered heterocyclyl or 5-10 membered heteroaryl is optionally substituted with one or more R⁷b;
      R⁷a is hydrogen, halogen, —CN, C1-6 alkyl, —OR⁷C, —NR⁷ cR⁷ ct, —C(O)R⁷C, —C(O)OR⁷C, —C(O)NR⁷ cR⁷d, —SO2R⁷C, —P(O)R⁷ cR⁷d, —SO2NR⁷ cR⁷d, —NR⁷ cC(O)R⁷d, —NR⁷ cC(O)OR⁷ ct, —NR⁷ cSO2R⁷ ct, —NR⁷ cSO2NR⁷ ctR⁷3-12 membered carbocyclyl,
   3-12 membered heterocyclyl, 6-10 membered aryl, or 5-10 membered heteroaryl, wherein said C1-6 alkyl, 3-12 membered carbocyclyl, 3-12 membered heterocyclyl, 6-10 membered aryl, or 5-10 membered heteroaryl represented by R⁷a is optionally substituted by one or more R⁷f;
      R⁷b is hydrogen, halogen, —CN, oxo, C1-6 alkyl, C2-6 alkenyl, C2-6 alkynyl, C1-6 alkoxy, —OR⁷c, —NR⁷ cR⁷ ct, —C(O)R⁷C, —C(O)OR⁷c, —C(O)NR⁷ cR⁷ ct, —SO2R⁷C, —P(O)R⁷ cR⁷ ct, —SO2NR⁷ cR⁷ ct, —NR⁷ cC(O)R⁷ ct, —NR⁷ cC(O)OR⁷ ct, —NR⁷ cSO2R¹ ct, —NR⁷ cSO2NR⁷dR⁷3-12 membered carbocyclyl, 3-12 membered heterocyclyl, 6-10 membered aryl, or 5-10 membered heteroaryl, wherein C1-6 alkyl, C2-6 alkenyl, C2-6 alkynyl, C1-6 alkoxy, 3-12 membered carbocyclyl, 3-12 membered heterocyclyl, or 5-10 membered heteroaryl represented by R⁷b is optionally substituted by one or more
   R1r;
      R⁷C, R⁷ ct, and R⁷e are independently selected from the group consisting of hydrogen, C1-6 alkyl, 3-12 membered carbocyclyl, 4-12 membered heterocyclyl, 6-10 membered aryl, and 5-10 membered heteroaryl; or R⁷c and R⁷d together with the N or P atom to which they are attached form 4-12 membered heterocyclyl or 5-10 membered heteroaryl; wherein said C1-6 alkyl, carbocyclyl, heterocyclyl, aryl, or heteroaryl represented by R⁷C, R⁷ ct, or R⁷e or in the group represented by R⁷C, R⁷ ct, or R⁷e is optionally substituted with one or more R⁷f;
      R⁷f, in each occurrence, is hydrogen, halogen, —CN, or —OH; and
   n is 0, 1, 2, or 3;
   wherein said heterocyclyl comprises 1-4 heteroatoms selected from O, N, and S; and said heteroaryl comprises 1-4 heteroatoms selected from O, N, and S.
(n) wo2022/058344 The compound according to formula (II)

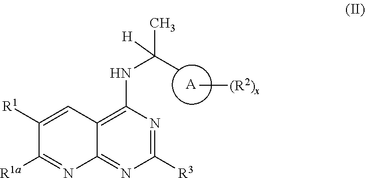

wherein
   A is phenyl;
      is selected from halogen, 5 to 10 membered mono or bicyclic heterocycloalkyl or heterocycloalkenyl with one or 2 nitrogen as heteroatoms and substituted by —CH3, —C(═O)—CH3 or —NH—C(═O)—CH3,
   R¹a is selected from hydrogen, —CH3, CF3 or —OCH3;
   R² is selected from hydrogen, halogen or C1-6-alkyl optionally one or more time substituted by halogen and/or hydroxyl;
   x is selected from 1 or 2 and is selected from hydrogen or —CH3;
   or a stereoisomer, a tautomer, an N-oxide, a hydrate, a solvate, or a salt thereof, or a mixture of same.
(o) WO2022/026465

A compound of Formula (I):

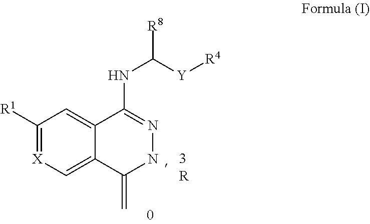

or a pharmaceutically acceptable salt thereof,
wherein:
R¹ is hydrogen, hydroxyl, C1-C6 alkyl, alkoxy, —N(R⁶)2, —NR⁶C(O)R⁶, —C(O)N(R⁶)2, —SO2 alkyl, —SO2NR⁶ alkyl, cycloalkyl, -Q-heterocyclyl, aryl, or heteroaryl, wherein the cycloalkyl, the heterocyclyl, the aryl, or the heteroaryl are each optionally substituted with one or more R²;
each Q is independently a bond, 0 or NR⁶;
X is N or CR⁷; with the proviso that when X is N, R¹ is not hydroxyl; each R² is independently hydroxy, halogen, cyano, hydroxyalkyl, haloalkyl, alkoxy, —N(R⁶)2, —SO2 alkyl, —NR⁶C(O)C1-C3 alkyl, —C(O)cycloalkyl, —C(O)heterocyclyl or aryl, wherein the cycloalkyl, the heterocyclyl or the aryl are each optionally substituted with one or more R⁹;
R³ is hydrogen, C1-C3 alkyl, C1-C3 haloalkyl, or cycloalkyl;
Y is a bond or heteroarylene;
R⁴ is aryl or heteroaryl, each optionally substituted with one or more R⁵;
each R⁵ is independently hydroxy, halogen, cyano, hydroxyalkyl, alkoxy, C1-C4 alkyl, haloalkyl, —N(R⁶)2, -L-N(R⁶)2 or —SO2 alkyl;
L is C1-C3 alkylene;
each R⁶ is independently hydrogen, C1-C3 alkyl, haloalkyl or cycloalkyl;
R⁷ is hydrogen, cyano or alkoxy;
R⁸ is C1-C2 alkyl or halo-C1-C2 alkyl; and
each R⁹ is independently C1-C3 alkyl or haloalkyl.

Each of the genus described in formulae (a)-(o) are preferred as individual genus.

The present invention provides for any individual genus or individual compound described in those genus for use in the treatment of pain and for use in the treatment of pain in combination with additional therapeutic agents.

The activity of a SOS1 inhibitor may be measured in the HTRF binding assay described in Hillig et al, PNAS| Feb. 12, 2019| vol. 116| no. 7| 2551-2560.

Other SOS1 assays are well known to the skilled person and include assays such as FRET/SPR binding.

Suitable SOS1 inhibitors for use in the present invention have an IC50's in the HTRF binding assay of less than or equal to 5 micromolar.

Particularly suitable SOS1 inhibitors have an IC50 of less than 100 nanomolar in the HTRF binding assay.

In a particularly preferred embodiment, the SOS1 inhibitors have an IC50 of 1 nanomolar or less in the HTRF binding assay.

The SOS1 inhibitors of the present invention also show selectivity for SOS1 over additional targets. Suitably, the SOS1 inhibitors of the present invention show selectivity of greater than or equal to 100 fold over one or more of the following targets: MEK 1, MEK 2, TrkA kinase, TrkB kinase, TrkC kinase, C—Raf, B-Raf, PI3 kinase, AKT and ERK.

When determining whether a compound of the present invention has a selectivity of more than a 100 fold for SOS1 over another target the following assays and methods may be used:

MEK 1 and 2 can be assayed using MEK assay kit, product code CS0490, Sigma, St Louis, USA.

Trk receptor kinase activity can be assayed as described in Wang et al, Curr Chem Genomics. 2008; 1: 27-33.

B-Raf can be assayed using the B-Raf Kinase Assay Kit, product code 17-359, Sigma, St Louis, USA.

C—Raf can be assayed using the BPS bioscience assay kit catalogue number 79570, San Diego, CA 92121. United States.

PI3 kinase can be assayed via the method described by Fry, Methods Mol Biol, 2009; 462:345-62.

AKT can be assayed using the abcam kit Akt Kinase Activity Assay Kit (ab139436), abcam plc, Cambridge, USA.

ERK can be assayed using the Promega ERK2 kinase kit, catalogue number V1961, Promega corporation, Madison, USA.

Specific SOS1 inhibitors suitable for use include:

WO/2022/170952 POLYCYCLIC PYRIDAZINONE DERIVATIVE SERVING AS SOS1 INHIBITOR, PREPARATION METHOD THEREFOR AND USE THEREOF

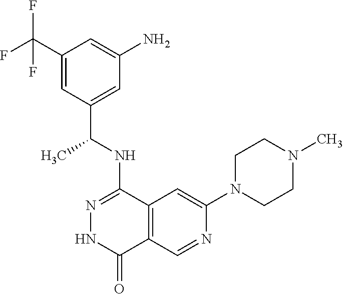 [SMILES: C[C@@H](Nc1n[nH]c(=O)c2cnc(N3CCN(C)CC3)cc12)c1cc(N)cc(C(F)(F)F)c1]

### Example 5

WO/2022/170802 PYRIMIDO-PYRIDONE DERIVATIVE AS SOS1 INHIBITOR, PREPARATION METHOD THEREFOR AND USE THEREOF

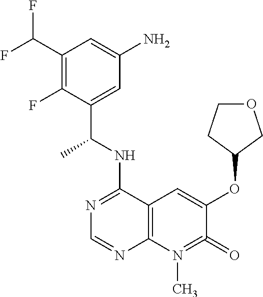

### Example 5

WO/2022/170917 POLYCYCLIC PYRIMIDINE DERIVATIVE AS SOS1 INHIBITOR, AND PREPARATION METHOD THEREFOR AND USE THEREOF

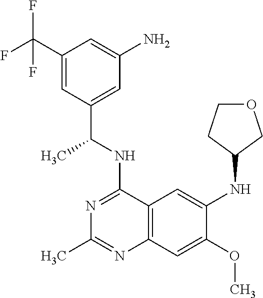

### Example 1

WO/2022/171018 SUBSTITUTED BENZOPYRIMIDINE OR PYRIDOPYRIMIDINE AMINE INHIBITOR, AND PREPARATION METHOD THEREFOR AND USE THEREOF

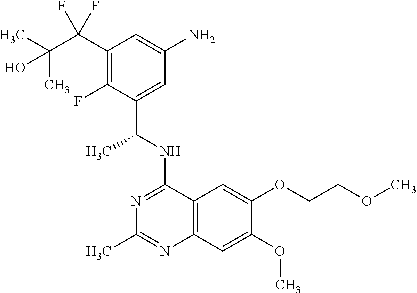 [SMILES: COCCOc1cc2c(N[C@H](C)c3cc(N)cc(C(F)(F)C(C)(C)O)c3F)nc(C)nc2cc1OC]

### Example 72

WO/2022/166974 PYRIDOPYRIMIDINONE DERIVATIVE, PREPARATION METHOD THEREFOR, AND USE THEREOF

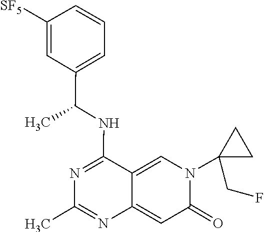

### Example 1-3

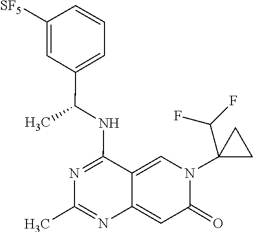

### Example 1-23

WO/2022/161461 SOS1 INHIBITOR, PREPARATION METHOD THEREFOR, AND APPLICATION THEREOF

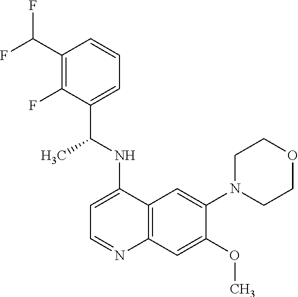 [SMILES: COc1cc2nccc(N[C@H](C)c3cccc(C(F)F)c3F)c2cc1N1CCOCC1]

### Example 2

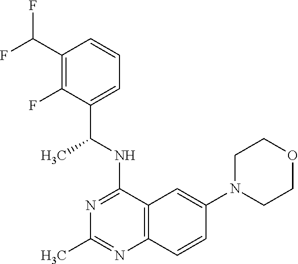 [SMILES: Cc1nc(N[C@H](C)c2cccc(C(F)F)c2F)c2cc(N3CCOCC3)ccc2n1]

### Example 12

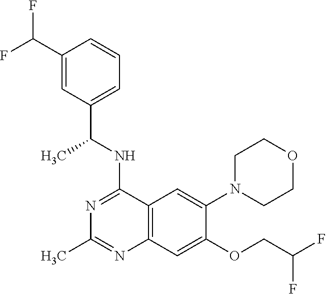 [SMILES: Cc1nc(N[C@H](C)c2cccc(C(F)F)c2)c2cc(N3CCOCC3)c(OCC(F)F)cc2n1]

### Example 47

WO/2022/148442 6-SUBSTITUTED PHOSPHORYL QUINAZOLINE DERIVATIVE, PREPARATION METHOD THEREFOR AND USE THEREOF

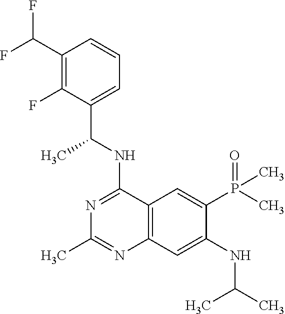 [SMILES: Cc1nc(N[C@H](C)c2cccc(C(F)F)c2F)c2cc(P(C)(C)=O)c(NC(C)C)cc2n1]

### Example 1-33

WO/2022/135610 TETRACYCLIC COMPOUND, PHARMACEUTICAL COMPOSITION THEREOF AND USE THEREOF

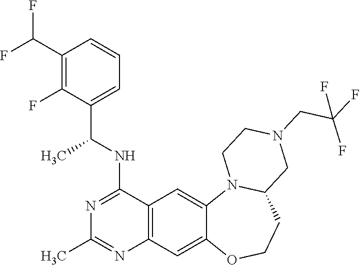 [SMILES: Cc1nc(N[C@H](C)c2cccc(C(F)F)c2F)c2cc3c(cc2n1)OCC[C@H]1CN(CC(F)(F)F)CCN31]

### Example 89

WO/2022/105921 PYRIMIDO-HETEROCYCLIC COMPOUND, PREPARATION METHOD THEREFOR, AND USE THEREOF

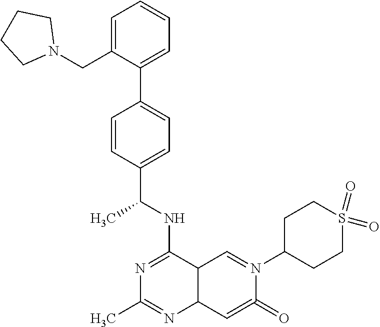

### Example 85

WO/2022/017339 FUSED PYRIDAZINE DERIVATIVE, PREPARATION METHOD THEREFOR AND PHARMACEUTICAL USE THEREOF

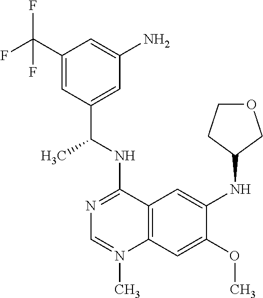

### Example 1

WO/2019/122129 NOVEL BENZYLAMINO SUBSTITUTED PYRIDOPYRIMIDINONES AND DERIVATIVES AS SOS1 INHIBITORS

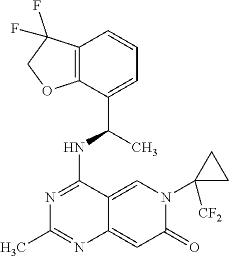

4-[[(1R)-1-(3,3-difluoro-2H-1-benzofuran-7-yl)ethyl]amino]-6-[1-(difluoromethyl)cyclopropyl]-2-methylpyrido[4,3-d]pyrimidin-7-one. Synthesis published in WO2019122129.

### Example 1-102

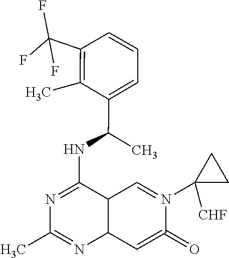

### Example 1-38

WO2022187411

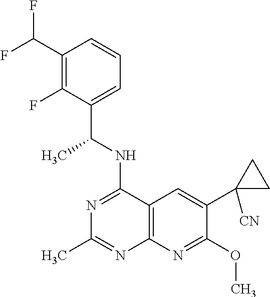 [SMILES: COc1nc2nc(C)nc(N[C@H](C)c3cccc(C(F)F)c3F)c2cc1C1(C#N)CC1]

### Example 184

Each of the individual compounds described above is a preferred embodiment of the present invention.

The SOS1 inhibitors were tested in an in vitro model of pain, on the NGF stimulated PC12 assay (Sasagawa et al, NATURE CELL BIOLOGY VOLUME 7, NUMBER 4, APRIL 2005, 365-373). The tested compounds showed great efficacy in the model.

The SOS1 inhibitors have numerous advantages as a pain treatment; they don't have the addiction potential of opiates and they show great efficacy. They also don't appear to have the side effects that make tanezumab and other anti-NGFs almost impossible to use at therapeutically effective doses.

In a further embodiment of the present invention, SOS1 inhibitors have been found to be particularly suitable for use in the treatment of pain when administered in combination with an anti NGF antibody.

The present invention provides a method of treating pain by administering a therapeutically effective amount of a SOS1 inhibitor as described herein in combination with an anti-NGF antibody.

Tanezumab is an example of an anti-NGF antibody. Its a promising and highly efficacious pain therapy, but patients frequently suffer unpleasant side effects at dosage levels sufficient to provide pain relief.

The combination provides a cooperative level of efficacy, with the advantage that the anti-NGF antibody can be administered at a dosage levels sufficient to provide pain relief without reaching a level where an adverse event may be seen. In cooperative systems, two independent agents are able to show a level of activity equivalent to one of the agents at a much higher dose. Its surprising to find two agents combining to have such an effect.

The scientific literature teaches that Tanezumab shows efficacy in rats at 10 mg/kg. (Miyagi et al Efficacy of nerve growth factor antibody in a knee osteoarthritis pain model in mice https://www.ncbi.nlm.nih.gov/pmc/articles/PMC5670727/Max efficacious dose of anti NGF is 10 mg/kg mouse Ghilardi et al Neuroplasticity of Sensory and Sympathetic Nerve Fibers in the Painful Arthritic Joint
https://www.ncbi.nlm.nih.gov/pmc/articles/PMC3386465/Max efficacious dose of anti NGF is 10 mg/kg mouse Shelton et al Nerve growth factor mediates hyperalgesia and cachexia in auto-immune arthritis https://pubmed.ncbi.nlm.nih.gov/15927377/Max efficacious dose of anti NGF is 10 mg/kg mouse)

Surprisingly, low doses of a SOS1 inhibitor in combination with 0.3 mg/kg Tanezumab has a significant analgesic effect.

The increase in analgesia with fixed dose SOS1 is related to the dose of Tanazumab (ie efficacy is dose related)

These data indicate that it should be possible to lower the dose of Tanezumab in humans and as a result reduce the propensity for side effects that limit use if of this class of drug. The combination of SOS1 inhibition with NGF blocking via monoclonal antibodies such as Tanezumab will deliver increased pain efficacy with reduced side effects when compared to the use of higher doses of Tanezumab alone.

Combinations of SOS inhibitors with NGF monoclonal antibodies, or other blockers/modulators of the NGF pathway, have the potential to deliver greater pain efficacy with reduced side effects leading to improved and enhanced treatment of pain in conditions such as osteoarthritis.

Accordingly, the present invention provides for the use of a SOS1 inhibitor as disclosed herein, in combination with an anti NGF, wherein one or both components is administered at a sub-therapeutic dose for the treatment of pain.

The term sub therapeutic dose is used to describe to describe a dose lower than that at which the component shows efficacy as a monotherapy.

Other advantages for the combination include the potential for oral dosing instead of intravenous or sub-cutaneous dosing. The combination may also result in a lower cost of treatment and provide a lower risk of immunogenicity.

Particularly suitable anti NGF antibodies include Tanezumab, Fasinumab, Fulranumab and MED1735.

Particularly suitable anti-NGF antibodies are Tanezumab and Fasinumab.

In a preferred embodiment the anti NGF antibody is Tanezumab.

In another preferred embodiment the anti NGF antibody is Fasinumab.

In a particularly preferred embodiment, the present invention provides for the use of a SOS1 inhibitor in combination with a sub therapeutic dose of Tanezumab, for the treatment of pain. Optionally, both the SOS1 inhibitor and Tanezumab are administered at a sub therapeutic dose.

The term sub therapeutic dose is used to describe to describe a dose lower than that at which the component shows efficacy as a monotherapy.

Suitable SOS1 inhibitors for use in the present invention have an IC50's in the HTRF binding assay of less than or equal to 5 micromolar.

Particularly suitable SOS1 inhibitors have an IC50 of less than 100 nanomolar.

In a particularly preferred embodiment the SOS1 inhibitors have an IC50 of 1 nanomolar or less.

The SOS1 inhibitors of the present invention also show selectivity for SOS1 over additional targets. Suitably, the SOS1 inhibitors of the present invention show selectivity of greater than or equal to 100 fold over one or more of the following targets: MEK 1, MEK 2, TrkA kinase, TrkB kinase, TrkC kinase, C—Raf, B-Raf, PI3 kinase, AKT and ERK. Without being bound by theory, its believed that SOS1 inhibitors act to treat pain in the following way:

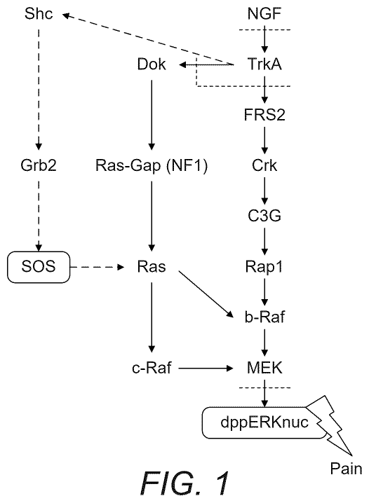

Nerve growth factor (NGF) is a protein that binds to the NGF receptor (TrkA), leading to the upregulation of genes involved in nociception. NGF is known to be an important contributor to the development of chronic pain. The NGF binding to TrkA and subsequent signal transduction culminates in the nuclear accumulation of diphopshorylated Extracellular signal-regulated kinase (dppERKnuc) in neurons, upregulating pain genes, as shown in FIG. 1.

Levels of SOS & molecules influenced by it downstream such as Ras feed into this cascade, with greater levels of SOS leading to higher levels of bRAF and MEK, leading to the accumulation of diphopshorylated Extracellular signal-regulated kinase (dppERKnuc), leading to higher levels of Pain. By inhibiting this pathway it is possible to control the formation of RAS GTP. By lowering levels of RAS GTP, pain is reduced.

The term pain includes but is not limited to: acute pain; chronic pain; inflammatory pain; nociceptive pain; neuropathic pain; hyperalgesia; allodynia; central pain; cancer pain; post-operative pain; visceral pain; musculo-skeletal pain; heart or vascular pain; head pain including migraine; orofacial pain, including dental pain; and back pain.

In more detail, suitable pain for treatment includes but is not limited to:
(a) acute pain and/or spontaneous pain,
(b) chronic pain and or on-going pain,
(c) inflammatory pain including any one of arthritic pain, pain resulting from osteoarthritis or rheumatoid arthritis, resulting from inflammatory bowel diseases, psoriasis and eczema
(d) nociceptive pain,
(e) neuropathic pain, including painful diabetic neuropathy or pain associated with post-herpetic neuralgia,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain, including digestive visceral pain and non-digestive visceral pain, pain due to gastrointestinal (GI) disorders, pain resulting from functional bowel disorders (FBD), pain resulting from inflammatory bowel diseases (IBD), pain resulting from dysmenorrhea, pelvic pain, cystitis, interstitial cystitis or pancreatitis,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, pyomyositis,
(m) heart or vascular pain, pain due to angina, myocardical infarction, mitral stenosis, pericarditis, Raynaud's phenomenon, scleredoma, scleredoma or skeletal muscle ischemia,
(n) head pain including migraine, migraine with aura, migraine without aura cluster headache, tension-type headache.
(o) orofacial pain, including dental pain, temporomandibular myofascial pain or tinnitus, or
(p) back pain, bursitis, menstrual pain, migraine, referred pain, trigeminal neuralgia, hypersensitisation, pain resulting from spinal trauma and/or degeneration or stroke.

Treatment of pain includes, but is not limited to, preventing, ameliorating, controlling, reducing incidence of, or delaying the development or progression of pain.

Particularly suitable pain indications include Osteoarthritis and cancer pain.

In another embodiment a suitable indication is osteoarthritis. According to another aspect of the invention there is provided the compounds of the present invention for separate, sequential or simultaneous use in a combination combined with a second pharmacologically active compound. Preferably the second pharmacologically active compound of the combination may include but is not limited to;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflunisal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butalbital, mephobarbital, metharbital, methohexital, pentobarbital, phenobarbital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorocyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphenadrine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR²B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g. oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2, 3, 6, 7, 12, 12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1 S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2—Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine; or
a 5-HT3 antagonist, such as ondansetron;
and the pharmaceutically acceptable salts and solvates thereof.

The invention further provides a pharmaceutical formulation comprising a compound of formula I, as defined above, or a pharmaceutically acceptable salt or solvate thereof, and a pharmaceutically acceptable adjuvant, diluent or carrier. The pharmaceutical formulation may further comprise one or more additional active agents for the treatment of a disorder mentioned above.

The invention further provides a pharmaceutical kit comprising a compound of formula I, as defined above, or a pharmaceutically acceptable salt or solvate thereof, and one or more additional active agents, as a combined preparation for separate, simultaneous or sequential administration in the treatment of a disorder mentioned above.

The invention further provides a method of treatment of a disorder mentioned above in a mammal (especially a human), comprising administration of a therapeutically effective amount of a compound of formula I, as defined above, or a pharmaceutically acceptable salt or solvate thereof, to a mammal in need of such treatment.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight 25% to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic) acid (PGLA) microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol.

Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™ Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 to 10,000 μg of the compound of the invention. The overall daily dose will typically be in the range 1 μg to 10 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of the compounds of the invention is typically in the range 0.5 mg to 3000 mg depending, of course, on the mode of administration. For example, oral administration may require a total daily dose of from 3 mg to 3000 mg, while an intravenous dose may only require from 0.5 mg to 500 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

### EXPERIMENTAL

### Materials and Methods.

### Identification of SOS1 Inhibitors:

The following method is based on the HTRF binding assay described in Hillig et al, PNAS Feb. 12, 2019, vol. 116, no. 7, 2551-2560

Measurement and evaluation of inhibition data, calculation of IC50 values.

Homogeneous time-resolved fluorescence (HTRF) was measured with a PHERAstar reader (BMG, Germany) using the HTRF module (excitation: 337 nm; emission 1: 620 nm, emission 2: 665 nm). The ratio of the emissions at 665 and 620 nm was used as the specific signal for further evaluation. The data were normalized using the controls: DMSO=0% inhibition, inhibition control wells with inhibitor control solution=100% inhibition. Compounds were tested in duplicates at up to 11 concentrations (e.g. 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated using a four-parameter fit, with a commercial software package (Genedata Screener, Switzerland). KRASG12C activation by SOS1 cat assay (“On-assay”). This assay quantifies SOS1 cat mediated loading of KRASG12C-GDP with a fluorescent GTP analogue. Detection of successful loading was achieved by measuring resonance energy transfer from anti-GST-terbium (FRET donor) bound to GST-KRASG12C to the loaded fluorescent GTP analogue (FRET acceptor). The fluorescent GTP analogue EDA-GTP-DY-647P1 [2′/3′-O-(2-aminoethyl-carbamoyl)guanosine-5′-triphosphate labelled with DY-647P1 (Dyomics GmbH, Germany)] was synthesized by Jena Bioscience (Germany) and supplied as a 1 mM aqueous solution. A KRASG12C working solution was prepared in assay buffer [10 mM HEPES pH 7.4 (AppliChem), 150 mM NaCl (Sigma), 5 mM MgCl2 (Sigma), 1 mM DTT (Thermo Fisher), 0.05% BSA Fraction V pH 7.0 (ICN Biomedicals), 0.0025% (v/v) Igepal (Sigma)] containing 100 nM GST-KRASG12C and 2 nM anti-GST-terbium (Cisbio, France). A SOS1 cat working solution was prepared in assay buffer containing 20 nM SOS1 cat and 200 nM EDA-GTP-DY-647P1. An inhibitor control solution was prepared in assay buffer containing 200 nM EDA-GTP-DY-647P1 without SOS1 cat. All steps of the assay were performed at 20° C. A volume of 2.5 μL of the KRASG12C working solution was added to all wells of the test plate using a Multidrop dispenser (Thermo LabSystems). After 10 min, 2.5 μL of the SOS1 cat working solution was added to all wells, except for the inhibitor control solution wells. After 30 min incubation, HTRF was measured.

The ability of SOS1 inhibitors to treat pain was measured using the assay below, based on the NGF stimulated PC12 assay (Sasagawa et al, NATURE CELL BIOLOGY VOLUME 7, NUMBER 4, APRIL 2005, 365-373).

Inhibition of Nerve Growth Factor (NGF) stimulated phospho-Extracellular Regulated Kinase 1 and 2 (pERK1/2) activation in the PC-12 cell line by SOS1 inhibitor.

### Homogeneous Time-Resolved Fluorescence (HTRF) Assay:

### SOS1 Inhibitor Inhibition at Different NGF Stimulus Concentrations:

The inhibitory effect of a selective, small molecule Rat sarcoma:Son of Sevenless1 (RAS:SOS1) inhibitor, was monitored via an HTRF readout measuring phosphorylation of ERK1/2 following NGF activation. All assays were performed in rat adrenal PC-12 cells (Merck) that had been serum-starved for a period of 24 hours in RPMI-1640 growth medium (Gibco) supplemented with 1% heat-inactivated horse serum (Merck), 0.5% heat-inactivated fetal bovine serum (FBS), 1% penicillin-streptomycin and 2 mM L-Glutamine, unless specified otherwise. Reagents from the HTRF commercial kit (Cisbio) were prepared according to the manufacturer's instructions.

### Assay Procedure:

PC-12 cells were isolated from routine cellular culture and plated at an appropriate cell density in 384-well plates (typically 25,000 cells per well) for 24 hours under serum-starved conditions. Following incubation, PC-12 cells were pre-treated with working concentrations of BI-3406 across an appropriate concentration response range for 30 minutes (37° C./5% CO₂). Duplicate concentration response curves for SOS1 Inhibitor were set-up per NGF (Merck) concentration tested. Following the 30-minute compound pre-incubation, PC-12 cells were treated with NGF (titrations of NGF from 250 ng/mL to 10 ng/mL were tested), and subsequently incubated for a 5-minute period (37° C./5% CO₂). Following 5 minutes of NGF treatment, lysis buffer from the commercial HTRF kit was applied to the PC-12 cells for 30-minutes of incubation with shaking (20° C. at 600 rpm). An appropriate volume of lysate was harvested per well and transferred to a separate 384-well Proxiplate (Perkin Elmer) to which a 5× concentrated HTRF kit antibody mix was dispensed into each lysate sample well. A 2-hour incubation at room temperature was performed prior to fluorescence signal determination using a microplate reader (PHERAstar FSX, BMG Labtech).

### Analysis.

i. SOS1 Inhibitor IC50 Plot Against NGF Activation:

Percentage inhibition values were calculated per compound concentration by normalising the sample data to the high and low controls used within each plate (+/−25 ng/mLNGF respectively). Percentage inhibition values from each duplicate SOS1 inhibitor concentration response curve were meaned to provide a data point per concentration. Mean percentage inhibition values were then used to determine the 50% inhibitory concentration (IC50) and maximum % efficacy (inhibition) was (maximum % efficacy SOS1 inhibitor/maximum % efficacy given by −NGF versus+NGF difference), using a four-parameter logistic fit where y=A xnH/(EC50nH+xnH), and x and y represent the test agent concentration and % cell pERK signal, respectively. The parameter EC50 is test agent concentration half-maximal output and A is the maximal inhibition (efficacy), while nH is the Hill coefficient (GraphPad Prism). Response data were then plotted against the molar logarithm for each SOS1 inhibitor compound concentration together with the determined fit results for display purposes. Error bars represent one standard deviation.

ii. SOS1 Inhibitor IC0 Versus NGF Concentration:

IC50 values were calculated for SOS1 inhibitor at NGF concentrations, 250 ng/mL, 200 ng/mL, 150 ng/mL, 100 ng/mL, 50 ng/mL, 25 ng/mL and 10 ng/mL following the analysis described in section “i”. The resulting geometric mean of the IC50 values (y-axis) per NGF concentration (x-axis) were subsequently plotted within a separate graph. Error bars represent one standard deviation calculated from 2-8 separate IC50 replicates.

iii. Percentage Maximum Efficacy Versus NGF Concentration:

Percentage inhibition values were calculated per compound concentration across an appropriate SOS1 inhibitor concentration response range as described in section ‘i’. The mean percentage inhibition value calculated for the top concentration of SOS1 inhibitor compound tested per NGF concentration was calculated from 2-8 replicates and subsequently plotted within a separate graph to show percentage maximum efficacy (y-axis) against NGF concentration (x-axis). Error bars represent one standard deviation.

Combination Work; Co-Operativity of antiNGF and SOS1 Inhibitor Inhibition.

The combined inhibitory effect of a selective, small molecule SOS1 inhibitor and known antibody based NGF inhibitor, Anti-NGF was monitored via an HTRF readout measuring phosphorylation of ERK1/2 by NGF activation. All assays were performed in rat adrenal PC-12 cells (Merck) that had been serum-starved for a period of 24 hours in RPMI-1640 growth medium (Gibco) supplemented with 1% heat-inactivated horse serum (Merck), 0.5% heat-inactivated fetal bovine serum (FBS), 1% penicillin-streptomycin and 2 mM L-Glutamine unless specified otherwise. Reagents from the HTRF commercial kit (Cisbio) were prepared according to the manufacturer's instructions.

### Assay Procedure:

PC-12 cells were isolated from routine cellular culture and plated at an appropriate cell density (typically 25,000 cells per well) in 384-well plates for 24 hours under serum-starved conditions. Working preparations of varying Anti-NGF (Abcam) concentrations and a fixed NGF concentration were prepared in serum-starved media and pre-incubated for 30 minutes (37° C./5% CO₂). Simultaneously, PC-12 cells were pre-treated with working concentrations of SOS1 inhibitor for 30 minutes (37° C./5% CO₂). Duplicate concentration response curves for SOS1 inhibitor were set-up per Anti-NGF; NGF combination tested. Following 30 minutes of pre-incubation, PC-12 cells were treated with an appropriate Anti-NGF; NGF combination (titrations of Anti-NGF ranging from 30 μg/mL to Opg/mL against a fixed 250 ng/mL concentration of NGF were tested), and subsequently incubated for a 5-minute period (37° C./5% CO₂). Following 5 minutes of Anti-NGF; NGF treatment, lysis buffer from the commercial HTRF kit was applied to the PC-12 cells for 30-minutes of incubation with shaking (20° C. at 600 rpm). An appropriate volume of lysate was harvested per well and transferred to a separate 384-well Proxiplate (Perkin Elmer) to which a 5× concentrated HTRF kit antibody mix was dispensed into each lysate sample well. A 2-hour incubation at room temperature was performed prior to fluorescence signal being measured in a microplate reader (PHERAstar FSX, BMG Labtech).

### Analysis.

i. SOS1 Inhibitor Percentage Efficacy Plot Against Anti-NGF; NGF Challenge:

Percentage inhibition values were calculated per compound concentration by normalising the sample data to the high and low controls used within each plate (+/−250 ng/mL NGF respectively). Percentage inhibition values from each duplicate SOS1 inhibitor concentration response curve were meaned to provide a data point per concentration. Mean percentage inhibition values were then used to determine the 50% inhibitory concentration (IC50) and maximum inhibition (efficacy) (maximum % inhibition (efficacy) SOS1 inhibitor/maximum % inhibition (efficacy) given by−NGF) and using a four-parameter logistic fit where y=A xnH/(EC50nH+xnH), where x and y represent the test agent concentration and % cell pERK signal, respectively. The parameter EC50 is test agent concentration half-maximal effect and A is the maximal output (efficacy), while nH is the Hill coefficient (GraphPad Prism). Response data were then plotted against the molar logarithm for each SOS1 inhibitor compound concentration together with the determined fit results for display purposes. Error bars represent one standard deviation. The mean percentage inhibition value calculated for both the top and bottom concentration of SOS1 inhibitor compound tested per Anti-NGF:NGF combination was extracted and compared in tabular form across the different Anti-NGF:NGF combinations tested.

### Western Blot:

### NGF Challenge Against SOS1 Inhibitor:

The inhibitory effect of a selective, small molecule SOS1 inhibitor was monitored via a Western Blot based readout (Jess, Protein Simple) measuring phosphorylation of ERK1/2 by NGF activation. All assays were performed in rat adrenal PC-12 cells (Merck) that had been serum-starved for a period of 24 hours in RPMI-1640 growth medium (Gibco) supplemented with 1% heat-inactivated horse serum (Merck), 0.5% heat-inactivated fetal bovine serum (FBS), 1% penicillin-streptomycin and 2 mM L-Glutamine unless specified otherwise. Reagents from the Jess Separation Module commercial kit (Protein Simple) were prepared according to the manufacturer's instructions.

### Assay Procedure:

PC-12 cells were isolated from routine cellular culture and plated at an appropriate cell density in 6-well plates for 24 hours under serum-starved conditions. Following incubation, PC-12 cells were pre-treated with working concentrations of SOS1 inhibitor for 30 minutes (37° C./5% CO₂). Duplicate concentration response curves for SOS1 inhibitor were set-up per NGF (Merck) concentration tested. Following the 30-minute compound pre-incubation, PC-12 cells were treated with an appropriate concentration of NGF, and subsequently incubated for a 5-minute period (37° C./5% CO₂). Following 5 minutes of NGF treatment, PC-12 cellular suspensions were transferred to falcon tubes and centrifuged (300×g for 5 minutes at 4° C.). Cellular supernatant was discarded, and the remaining cell pellet was washed with of ice-cold PBS. This was repeated for 3×PBS washes, before ice-cold lysis buffer containing protease and phosphatase inhibitors was added to the dry cell pellet. Tubes were subsequently sonicated for 30-seconds and stored on ice for 15-minutes with regular inversion to ensure completion of cellular lysis. Following lysis, samples were centrifuged (13,000×g for 5 minutes at 4° C.), and supernatant extracted to be prepared for Western Blot analysis. Supernatant samples were prepared for loading into Jess capillary cassettes according to the manufacturer's instructions. Duplicate samples per SOS1 inhibitor concentration tested were loaded within the same run. Data were analysed as described in i.

### Cell Culture and Growth-Factor Treatments.

PC12 cells (1) were purchased from the American Type Culture Collection (Rockville, MD) and cultured in RPMI-1640 (Biowhittaker, Walkersville, MD) with 10% horse serum (Life Technologies, Grand Island, NY) and 5% fetal bovine serum (Hyclone, Logan, UT). Cell viability was assessed by trypan blue dye exclusion. Prior to assays, cells were starved in DMEM for 16 h, then stimulated with Nerve Growth Factor (NGF-β; mouse submaxillary glands, Sigma, St. Louis, MO) which was dissolved in RPMI-1640 at the concentration of ng/μl and then diluted to the appropriate concentration before use.

### Immunofluorescence Assays.

Cells were plated on poly-L-lysine-coated coverslips or gridded glass-bottomed dishes, serum starved and stimulated with the indicated concentrations of NGF. Cells were fixed with 4% paraformaldehyde for 10 min at room temperature, permeabilized in 0.2% Triton X-100 for 10 min at room temperature or 100% methanol for 10 min at −20° C. and then blocked with 1% BSA for 30 min at room temperature. Cells were then incubated with primary antibodies anti-phospho-ERK1/2 (1:200) antibody for 1-2 h at RT, followed by secondary antibody (Alexa Fluor 647 anti-mouse IgG (1:500)) antibodies for 1 h at room temperature. All antibodies were supplied by Cell Signalling Technology, Danvers, MA, USA. Following equilibration, cells were then washed and the Fluorescence was measured using a spectrofluorometer (Molecular Devices Spectramax, San Jose, CA 95134 USA).

### Test Compounds.

Anti NGF monoclonal antibodies were supplied by Abcam (Cambridge, MA, USA). BI-3406 was supplied by MedchemExpress, NJ, USA.

### Data Analysis.

Data were Analysed Using GraphPad Prism.

### Results

A number of structurally diverse SOS1 inhibitors were tested in the Inhibition of Nerve Growth Factor (NGF) stimulated phospho-Extracellular Regulated Kinase 1 and 2 (pERK1/2) activation in the PC-12 cell line assay against a suitable control. All the tested compounds have an IC50 in SOS1 assay below 5.

All showed a level of efficacy between 40-70%.

The best compound had an IC50 of 10-20 nM

The ability of an NGF inhibitor Tanezumab in combination with a SOS1 inhibitor to treat pain was measured in the Inhibition of Nerve Growth Factor (NGF) stimulated phospho-Extracellular Regulated Kinase 1 and 2 (pERK1/2) activation in the PC-12 cell line assay.

At a concentration of 200 nM, tanezumab had 100% efficacy

At a concentration of 50 nM, Tanezumab had 90% efficacy.

SOS1 inhibitor at a concentration of 500 nM had 40% efficacy

Tanezumab at a concentration of 50 nM, in combination with SOS1 inhibitor at a concentration of 500 nM had ˜96% efficacy

1. A SOS1 inhibitor for use in the treatment of pain, wherein the SOS1 inhibitor is selected from:
(a) A compound represented by the general formula (I) or its tautomer, meso, racemate, enantiomer, diastereomer, or its mixture form, or its Medicinal salt:
in: 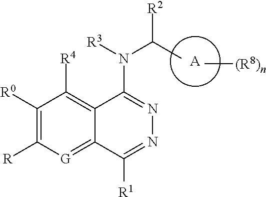
Ring A is aryl or heteroaryl;
G is CR⁵ or N atom;
R⁰ is selected from halogen, alkyl, alkoxy, haloalkyl, haloalkoxy, hydroxyalkyl, alkenyl, alkynyl, hydroxy, amino, —(CH2)pNR6R⁷, cycloalkyloxy, heterocyclyloxy, cycloalkyl, heterocyclyl, aryl and heteroaryl, wherein said alkyl, cycloalkyloxy, heterocyclyloxy, cycloalkyl, heterocyclyl, aryl and heteroaryl are each independently optionally selected from halogen, alkyl, haloalkyl, hydroxy, hydroxyalkyl, alkoxy, haloalkoxy, oxo, ═NH, amino, nitro, cyano, —S(O)2R⁹, —C(O)R10, cycloalkyl, heterocyclyl, aryl and heteroaryl are substituted with one or more substituents;
R1 is selected from hydrogen atom, halogen, alkyl, haloalkyl, alkoxy, haloalkoxy, hydroxyalkyl, cyano and cycloalkyl;
R2 is selected from halogen, alkyl, haloalkyl, hydroxyalkyl, hydroxy, cyano, cycloalkyl and heterocyclyl, wherein each of said alkyl, cycloalkyl and heterocyclyl is independently optionally selected Substituted from one or more substituents of halogen, alkyl, haloalkyl, hydroxy, hydroxyalkyl, alkoxy, haloalkoxy, amino, nitro and cyano;
R3 is selected from hydrogen atom, alkyl, haloalkyl, hydroxyalkyl, cycloalkyl, heterocyclyl, aryl and heteroaryl, wherein said alkyl, haloalkyl, hydroxyalkyl, cycloalkyl, hetero Cyclic, aryl, and heteroaryl are each independently optionally selected from halogen, alkyl, alkenyl, alkynyl, alkoxy, haloalkyl, haloalkoxy, cyano, amino, nitro, hydroxy, substituted with one or more substituents of hydroxyalkyl, cycloalkyl, heterocyclyl, aryl and heteroaryl;
R4 is selected from hydrogen atom, halogen, alkyl, haloalkyl, alkoxy, haloalkoxy, hydroxy, hydroxyalkyl and —(CH2)pNR6R7;
R and R5 are the same or different, each independently selected from hydrogen atom, halogen, alkyl, alkoxy, haloalkyl, haloalkoxy, alkenyl, alkynyl, cycloalkyl, heterocyclyl, aryl, heteroaryl group, cycloalkyloxy, heterocyclyloxy, aryloxy, heteroaryloxy, —(CH2)pNR6R7, cyano and nitro, wherein said alkyl, alkenyl, alkynyl, Cycloalkyl, heterocyclyl, aryl and heteroaryl are each independently optionally selected from alkyl, haloalkyl, alkoxy, haloalkoxy, halogen, cyano, nitro and —(CH2)qNR11R12 substituted by one or more of the substituents in;
R8 is the same or different, each independently selected from halogen, alkyl, alkenyl, alkynyl, haloalkyl, alkoxy, haloalkoxy, cyano, amino, —(CH2)pNR6R⁷, nitro, hydroxy, hydroxyalkane Alkyl, —S(O)2 alkyl, cycloalkyl, heterocyclyl, aryl and heteroaryl, wherein said alkyl, haloalkyl, hydroxyalkyl, cycloalkyl, heterocyclyl, aryl and heteroaryl are each independently optionally selected from hydroxy, halogen, haloalkyl, alkoxy, haloalkoxy, cyano, nitro, hydroxyalkyl, —(CH2)qNR11R¹², cycloalkyl, heterocycle substituted with one or more substituents in aryl, aryl and heteroaryl;
R9 and R10 are the same or different, each independently selected from hydrogen atom, alkyl, haloalkyl, hydroxyalkyl, —(CH2)qNR11R12, cycloalkyl and heterocyclyl, wherein said alkyl, cycloalkyl and The heterocyclyl groups are each independently optionally substituted with one or more substituents selected from the group consisting of hydroxy, halo, alkyl, haloalkyl, hydroxyalkyl, alkoxy, haloalkoxy, cyano, amino, and nitro;
R6, R7, R11 and R12 are the same or different and are each independently selected from hydrogen atoms, alkyl groups, haloalkyl groups, hydroxyalkyl groups, cycloalkyl groups, heterocyclyl groups, aryl groups and heteroaryl groups;
p and q are the same or different, each independently selected from 0, 1 and 2;
n is selected from 0, 1, 2, 3, 4 and 5.
(b) A pyrimido heterocyclic compound as shown in general formula I, or a pharmaceutically acceptable salt thereof, or its enantiomer, diastereomer, tautomer, twist isomer, solvates, polymorphs or prodrugs, 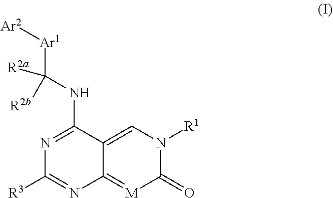
where:
R1 is independently selected from C1-C6 alkyl, C1-C6 haloalkyl, C2-C6 alkenyl, C2-C6 alkynyl, C3-C12 cycloalkyl, C4-C12 cycloalkenyl, 3-12 membered heterocycloalkane base, 5-12-membered aryl or 5-12-membered heteroaryl, carbocyclic or heteroatom-containing spiro/bridged/fused ring, wherein the C1-C6 alkyl, C2-C6 alkenyl, C2-C6 alkynyl, C3-C12 cycloalkyl, C4-C12 cycloalkenyl, 3-12 membered heterocycloalkyl, 5-12 membered aryl or 5-12 membered heteroaryl, carbocyclic or heterocyclic The spiro/bridged/fused ring of atoms can be optionally substituted by 1-3 Rn; or the above two Rn can form 3-12-membered saturated or partially unsaturated, or aromatic through carbon chains or heteroatoms Ring system; said Rn is selected from hydrogen, deuterium, halogen, cyano, nitro, amide, sulfonamide, hydroxyl, amino, urea, phosphoryl, alkyl phosphoroxy, alkylsilyl, C1-C6 Alkyl, C1-C6 alkoxy, haloalkyl, haloalkoxy, C1-C6 monoalkylamino, C1-C6 dialkylamino, alkenyl, alkynyl, 3-8 membered cycloalkyl or heterocycloalkane base, C1-C6 alkyl-S—, C1-C6 alkyl-SO—, C1-C6 alkyl-SO2—;
R2a and R2b are each independently selected from hydrogen, deuterium, halogen, C1-C6 alkyl, 3-8 membered cycloalkyl or heterocycloalkyl; and R2a and R2b or the substituent Rm on R2a and Ar may pass through a carbon chain Or heteroatoms form a 3-6 membered saturated or partially unsaturated or unsaturated ring system;
R3 is H, deuterium, halogen, hydroxyl, amino, cyano, C1-C6 alkyl, C1-C6 haloalkyl, C1-C6 alkoxy, C1-C6 haloalkoxy, C1-C6 alkylamino, 3-8-membered cycloalkyl or heterocycloalkyl, C2-C4 alkenyl, C2-C4 alkynyl, 5-10-membered aromatic ring or aromatic heterocyclic group;
M is independently selected from N or CR4, and R4 is selected from hydrogen, deuterium, halogen, cyano, C1-C6 alkyl, 3-8 membered cycloalkyl or heterocycloalkyl;
Ar1 and Ar2 are independently selected from 5-12-membered monocyclic or bicyclic aryl or heteroaryl groups, which may be substituted by one or more groups Rm selected from the group consisting of hydrogen: deuterium, halogen, cyano, nitro, substituted or unsubstituted amide, substituted or unsubstituted sulfonamide, hydroxyl, amino, urea, phosphoryl, alkylphosphooxy, alkylsilyl, C1-C10 Alkyl, C1-C10 alkoxy, C1-C10 alkoxyalkyl, C1-C10 haloalkyl, C1-C10 haloalkoxy, C1-C10 haloalkoxyalkyl, C1-C10 monoalkylamino, C1-C10 dialkylamino, C1-C10 monoalkylaminoalkyl, C1-C10 dialkylaminoalkyl, C1-C10 alkenyl, C1-C10 alkynyl, 3-12 membered cycloalkyl or heterocycloalkane base, 3-12 membered cycloalkyl or heterocycloalkylalkyl, C1-C10 alkyl-S—, C1-C10 alkyl-SO—, C1-C10 alkyl-SO2—, substituted or unsubstituted 5-12-membered aryl or heteroaryl, or the above two Rm can form a 3-12-membered saturated or partially unsaturated, or aromatic ring system through carbon chains or heteroatoms;
One or more hydrogen atoms on any of the above-mentioned groups can be substituted by a substituent selected from the following group: including but not limited to deuterium, halogen, C1-C3 alkyl, 3-6 membered cycloalkyl or heterocycloalkane wherein, the heteroaryl group contains 1-3 heteroatoms selected from the following group: N, O, P or S, and the heterocycloalkyl group contains 1-3 heteroatoms selected from the following group: N, O, P or S, the ring system includes spiro, bridged, fused, and saturated or partially unsaturated ring systems.
(c) 6-substituted phosphorylquinazoline derivatives represented by formula I, 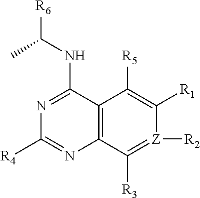
their tautomers, stereoisomers, hydrates, solvates, pharmaceutically acceptable salts or prodrugs:
in,
Z is a carbon atom or a nitrogen atom; and when Z is a nitrogen atom, R2 is absent;
R1 is
R11 and R12 are each independently C1-C6 alkyl or C1-C6 alkoxy; the C1-C6 alkyl or the C1-C6 alkoxy is independently substituted by one or more R13, the R13 is a substituent selected from the following: hydroxyl, amino, nitro, halogen, cyano; when there are multiple substituents, the substituents are the same or different;
Or, R11, R12 together with the phosphorus atom they are attached to form a substituent wherein, ring B is a 4-8 membered carbocyclic ring, a 4-8 membered alkene ring or a 4-8 membered heterocyclic ring;
Ra is independently hydrogen or a substituent selected from the group consisting of hydroxyl, amino, nitro, halogen, cyano, C1-C6 alkyl, 3-8 membered cycloalkyl; the C1-C6 alkyl, or the The 3-8 membered cycloalkyl groups are independently substituted by one or more Rf; when there are multiple substituents, the Rf is the same or different;
m is 1, 2, 3 or 4;
R2 is hydrogen or a substituent selected from the following: hydroxyl, amino, nitro, halogen, cyano,
wherein Y is absent or a group selected from:
R21, R22, R23, R24 are each independently hydrogen or a substituent selected from the group consisting of: C1-C6 alkyl, 3-8 membered cycloalkyl, 4-8 membered heterocycloalkyl, C2-C6 alkenyl, C2-C6 alkynyl, C1-C6 alkoxy; the C1-C6 alkyl, the 3-8 membered cycloalkyl, the 4-8 membered heterocycloalkyl, the C2-C6 alkenyl, the The C2-C6 alkynyl group or the C1-C6 alkoxy group is independently substituted by one or more Rf; when there are multiple substituents, the Rf is the same or different;
L is a group absent or selected from the group consisting of C1-C6 alkyl, deuterated C1-C6 alkyl, 3-8 membered cycloalkyl, 4-8 membered heterocycloalkyl, C2-C6 alkenyl, C2-C6 alkynyl, C1-C6 alkoxy; the C1-C6 alkyl, the 3-8 membered cycloalkyl, the 4-8 membered heterocycloalkyl, the C2-C6 alkenyl, the The C2-C6 alkynyl group or the C1-C6 alkoxy group is independently substituted by one or more Rf; when there are multiple substituents, the Rf is the same or different;
M is a substituent that is absent or selected from the group consisting of hydroxyl, amino, nitro, halogen, cyano, C1-C6 alkyl, C1-C6 alkoxy, —C00C1-C6 alkyl; the C1-C6 alkane group, the C1-C6 alkoxy group, or the —C00C1-C6 alkyl group are independently substituted by one or more Rf; when there are multiple substituents, the Rf is the same or different;
Ring A is absent or selected from 3-15-membered cycloalkyl or 4-15-membered heterocycloalkyl, 5-15-membered aryl or 5-15-membered heteroaryl;
Rb is independently hydrogen or a group selected from the group consisting of hydroxyl, amino, nitro, halogen, cyano, _NER5-C1-C6 alkyl, C1-C6 alkoxy, C2-C6 amido, C1-C6 ester group, C1-C6 carbonyl group; the C1-C6 alkyl group, the C1-C6 alkoxy group, the C2-C6 amido group, the C1-C6 ester group, or the C1-C6 carbonyl group independently be substituted by one or more Rf; when there are multiple substituents, the Rf is the same or different; n is 1, 2, 3 or 4; when there are multiple Rbs, Rb is the same or different substituents;
R3 is hydrogen or a substituent selected from the group consisting of halogen, cyano, C1-C6 alkyl, C1-C6 alkoxy, 3-8 membered cycloalkyl, 4-8 membered heterocycloalkyl; the C1-C6 alkyl, the C1-C6 alkoxy, the 3-8 membered cycloalkyl, or the 4-8 membered heterocycloalkyl are each independently substituted with one or more Rf; when there are multiple substituents, the Rf is the same or different;
R4 is hydrogen or a substituent selected from the group consisting of halogen, C1-C6 alkyl, C1-C6 alkoxy, 3-8 membered cycloalkyl; the C1-C6 alkyl, the C1-C6 alkoxy, the 3-8 membered cycloalkyl is independently substituted by one or more Rf; when there are multiple substituents, the Rf is the same or different;
R5 is hydrogen or a substituent selected from the group consisting of halogen, C1-C6 alkyl, C1-C6 alkoxy; said C1-C6 alkyl or said C1-C6 alkoxy are independently replaced by one or more Rf is substituted; when there are multiple substituents, the Rf is the same or different;
R6 is a 5-8 membered cycloalkyl group, or a 5-8 membered aromatic ring group, or a 5-membered heteroaromatic ring group; the 5-8 membered cycloalkyl group, or the 5-8 membered aromatic ring group, Or a 5-10 membered heteroaryl ring is optionally substituted with one or more, the same or different substituents selected from the group consisting of: hydroxyl, amino, nitro, halogen, cyano, —SF5, C1-C6 alkyl, C2-C6 alkenyl, C2-C6 alkynyl, C1-C6 alkoxy, 3-8-membered cycloalkyl, 4-10-membered heterocycloalkyl, —SO2-C1-C6 alkyl; the hydroxyl, Amino is optionally substituted by C1-C6 alkyl, 3-8-membered cycloalkyl or 4-10-membered heterocycloalkyl; wherein, the C1-C6 alkyl, C2-C6 alkenyl, C2-C6 alkynyl, C1-C6 alkoxy, 3-8-membered cycloalkyl, 4-10-membered heterocycloalkyl are independently substituted by one or more Rf; when there are multiple substituents, the Rf is the same or different;
The Rf is a substituent selected from the group consisting of hydroxyl, amino, nitro, halogen, cyano, C1-C6 alkyl, C1-C6 haloalkyl, C1-C6 alkoxy, C1-C6 haloalkoxy, 3-8-membered cycloalkyl, 3-8-membered halogenated cycloalkyl, 4-10-membered heterocycloalkyl, C1-C6 acyl, C1-C6 carbonyl, C1-C6 sulfone, C1-C6 halogenated sulfone;
In the heterocycloalkyl or heteroaryl ring group, the heteroatom is selected from one or more of N, O, S and P, and the number of heteroatoms is 1-3.
(d) A compound of formula (I) [symbol]
1. [symbol] (1) [symbol] [symbol] [symbol] 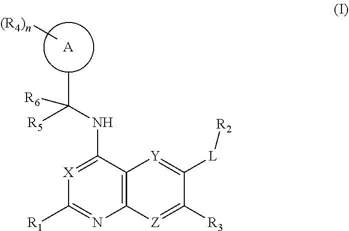
or a pharmaceutically acceptable salt thereof,
in,
X is selected from CH or N;
Y is selected from CH or N;
Z is selected from CH or N;
R1 is selected from H, CN, C1-6 alkyl or C3-6 cycloalkyl;
Ring A is selected from C6-10 aryl, benzo 5-7 membered heterocyclyl or benzo 5-7 membered heteroaryl;
L is selected from chemical bonds or 0;
R2 is selected from C3-10 cycloalkyl, C6-10 aryl, 3-10-membered heterocyclyl or 5-10-membered heteroaryl, the C3-10 cycloalkyl, C6-10 aryl, 3-10 A membered heterocyclyl or 5-10 membered heteroaryl is optionally substituted with R2b and/or R2c;
R2b is selected from —OR2c, —N(R2c)2, halogen, hydroxyl, cyano, amino, —C(O)R2c, —C(O)NHR2c, —C(O)NH2, —NHR2c, —C(O)H, —C(O)OH, —S(O)2NHR2c, —NHC(O)H, —N(C1-4 alkyl)C(O)H, —C(O)N(R2c)2, —C(O)OR2c, —S(O)2R2c, —S(O)2N(R2c)2, —NHC(O)R2c or —N(C1-4 alkyl)C(O)R2c;
R2c is independently selected from C1-6 alkyl, C1-3 deuterated alkyl, C3-10 cycloalkyl, C6-10 aryl, 3-10 membered heterocyclyl or 5-10 membered heteroaryl, the C1-6 alkyl, C3-10 cycloalkyl, C6-10 aryl, 3-10 membered heterocyclyl, 5-10 membered heteroaryl optionally substituted by R2d;
R2d is selected from halogen, hydroxyl, cyano, amino, —C(O)R2f, —C(O)N(R2f)2, —C(O)OR2f, —S(O)2R2f, —S(O)2N(R2f)2, —N(C1-4 alkyl)R2f, —NHC(O)R2f or —N(C1-4 alkyl)C(O)R2f;
R2f is independently selected from H or C1-6 alkyl;
R3 is selected from H, halogen, hydroxyl, cyano, amino, —NH—C3-6 cycloalkyl, C1-3 deuterated alkyl, —O—C1-3 deuterated alkyl, C1-6 alkyl, C3-6 cycloalkyl, 3-8 membered heterocyclyl, —O—C1-6 alkyl, —O—C3-6 cycloalkyl, —O-(3-8 membered heterocyclyl), 5-10 membered Heteroaryl, —C(O)R3a, —C(O)N(R3a)2, —C(O)OR3a, —S(O)2R3a, —S(O)2N(R3a)2, —NHC(O)R3a or —N(C1-4 alkyl)C(O)R3a, the —NH—C3-6 cycloalkyl, C1-6 alkyl, C3-6 cycloalkyl, 3-8 membered heterocycle group, —O—C1-6 alkyl, —O—C3-6 cycloalkyl, —O-(3-8 membered heterocyclyl) or 5-10 membered heteroaryl optionally substituted by R3b;
The R3a is independently selected from H or C1-6 alkyl;
The R3b is independently selected from halogen, hydroxyl, cyano, amino, 3-8 membered heterocyclyl or C1-6 alkyl;
R4 is selected from halogen, hydroxyl, cyano, amino, C1-6 alkyl, C3-6 cycloalkyl, —O—C1-6 alkyl, —O—C3-6 cycloalkyl, —O-(3-8-membered heterocyclyl), 3-8 membered heterocyclyl, 5-10 membered heteroaryl or —S(O)2-C1-4 alkyl, the C1-6 alkyl, C3-6 cycloalkyl, —O—C1-6 alkyl, —O—C3-6 cycloalkyl, —O-(3-8 membered heterocyclyl), 3-8 membered heterocyclyl or 5-10 membered heteroaryl optional is substituted by R4a; said R4a is selected from halogen, hydroxy, cyano or amino;
R5 is selected from C1-3 deuterated alkyl, C1-6 alkyl or C1-6 haloalkyl;
R6 is selected from H, deuterium, C1-3 deuterated alkyl, C1-6 alkyl or C1-6 haloalkyl;
n is selected from 0, 1, 2, 3 or 4;
Wherein, when X is selected from N, Z is selected from CH, R is selected from CN or C3-6 cycloalkyl;
When X and Z are selected from N, R1 is selected from CN, C1-6 alkyl or C3-6 cycloalkyl;
When X and Z are selected from CH, R3 is selected from hydroxyl, cyano, amino, C1-3 deuterated alkyl, —O—C1-3 deuterated alkyl, C1-6 alkyl, C3-6 cycloalkyl, 3-8 membered heterocyclyl, —O—C1-6 alkyl, —O—C3-6 cycloalkyl, —O-(3-8 membered heterocyclyl), 5-10 membered heteroaryl, —C(O)R3a, —C(O)N(R3a)2, —C(O)OR3a, —S(O)2R3a, —S(O)2N(R3a)2, —NHC(O)R3a or —N(C1-4 alkyl)C(O)R3a, the C1-6 alkyl, C3-6 cycloalkyl, 3-8 membered heterocyclyl, —O—C1-6 alkyl, —O—C3-6 cycloalkyl, —O-(3-8 membered heterocyclyl) or 5-10 membered heteroaryl optionally substituted with R3b.
(e) Pyridopyrimidone derivatives represented by formula I, 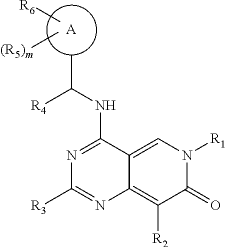
their tautomers, stereoisomers, hydrates, solvates, pharmaceutically acceptable salts or prodrugs:
Wherein, ring A is a 6-10-membered aromatic ring or a 9-11-membered heteroaromatic ring;
R1 is a 3-10 membered cycloalkyl or a 4-10 membered heterocycloalkyl, the R1 is optionally substituted with one or more R11, and the R11 is a substituent selected from the group consisting of halogen, hydroxyl, C1-C6 alkyl, C1-C6 haloalkyl, C1-C6 alkoxy, When there are multiple substituents R11, the substituents R11 are the same or different;
The R11 is optionally substituted with a substituent selected from the group consisting of C1-C6 alkyl, C1-C6 alkoxy, halogen, hydroxyl; R12 is C1-C6 alkyl, one or more F-substituted C1-C6 Alkyl or 3-6 membered cycloalkyl;
R13 is hydrogen, C1-C6 alkyl or cyano;
R14 is hydrogen, C1-C6 alkyl, C1-C6 haloalkyl;
R2 is hydrogen or a substituent selected from the following: halogen, C1-C6 alkyl, 3-6 membered cycloalkyl, C1-C6 alkoxy; the C1-C6 alkyl, 3-6 membered cycloalkyl, C1-C6 alkoxy is independently substituted by one or more R21; the R21 is a substituent selected from the following: hydroxyl, halogen, C1-C3 alkoxy; when there are multiple substituents, the R21 same or different;
R3 is hydrogen or a substituent selected from the group consisting of halogen, C1-C6 alkyl, C1-C6 haloalkyl;
R4 is C1-C6 alkyl or C1-C6 haloalkyl;
R5 is hydrogen or a substituent selected from the group consisting of halogen, C1-C6 alkyl, C1-C6 haloalkyl;
R6 is —SF5,
described R61, described R62 are each independently halogen-substituted C1-C6 alkyl or 3-6 membered cycloalkyl;
Or ring A and R6, R5 together form group fragment
wherein, Z is R63 is hydrogen or a substituent selected from the following: halogen, hydroxyl, C1-C6 alkyl, halogen substituted C1-C6 alkyl; When the substituent R63 is multiple, the R63 is the same or different;
m is 1 or 2; p is 1, 2 or 3; n is 1, 2 or 3.
(f) A polycyclic pyrimidine derivative, its pharmaceutically acceptable salt, its tautomer or its stereoisomer, characterized in that the structure of said polycyclic pyrimidine derivative is as shown in formula (I) 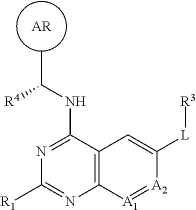
Wherein: R1 is selected from hydrogen or C1-C3 alkyl; preferably hydrogen or methyl;
A1 is selected from N or C—R11;
R11 is selected from H, C1-C3 alkyl or C1-C3 haloalkyl;
A2 is selected from N or C—R2;
R2 is selected from —OR21 or cyano;
R21 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R22;
R22 is selected from halogen, C1-C4 alkyl, cyano, hydroxyl;
L is absent or selected from O, NH or NCH3;
R3 is selected from 3-7 membered cycloalkyl, 4-7 membered heterocyclyl, 6-10 membered fused heterocyclyl, 6-10 membered bridged heterocyclyl, 6-10 membered spiro heterocyclyl, wherein 3-7 membered cycloalkyl, 4-7 membered heterocyclyl, 6-10 membered fused heterocyclyl, 6-10 membered bridged heterocyclyl, 6-10 membered spiro heterocyclyl optionally—3 R31 replaced;
R31 is selected from C1-C3 alkyl, C1-C3 haloalkyl, hydroxy, halogen, cyano, —NRaRb, C1-C3 alkoxy, ═O, —NHCOR32 or —COR32;
R32 is selected from C1-C3 alkyl, C1-C3 haloalkyl, 3-7 membered cycloalkyl or 4-7 membered heterocyclyl;
R4 is —CH3;
AR is selected from 6-10-membered aryl or 5-10-membered heteroaryl, wherein the aryl or heteroaryl is optionally substituted by 1-4 R5;
R5 is selected from halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —ORa, —NRaRb;
Ra is selected from H, C1-C4 alkyl, C1-C4 haloalkyl, 3-6 membered cycloalkyl or 4-7 membered heterocyclyl;
Rb is selected from H, C1-C4 alkyl, C1-C4 haloalkyl, 3-6 membered cycloalkyl or 4-7 membered heterocyclyl;
In the above definition, the following conditions cannot occur at the same time:
1) A1 is C—R11;
2) A2 is C—OR21;
3) L is 0 or does not exist;
The heteroatoms in the heterocyclic group and the heteroaryl group in the formula (I) are 1-3 and are selected from one or more of oxygen, nitrogen and sulfur.
(g) Substituted benzo or pyridopyrimidine amine compounds with general formula (I), their stereoisomers, tautomers, crystal forms, pharmaceutically acceptable salts, hydrates, solvates or prodrugs:
In the formula, [symbol]
1 [symbol] (1) [symbol] [symbol] [symbol] [symbol] [symbol] [symbol] [symbol] [symbol] [symbol] [symbol] 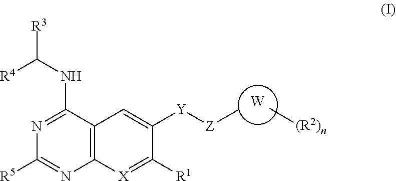
substituted or unsubstituted: C1-C6 alkyl, C3-C6 cycloalkyl or 4-6 membered heterocyclyl;
Z is selected from the group consisting of substituted or unsubstituted: bond, substituted or unsubstituted C1-C18 alkylene;
W is selected from the group of substituted or unsubstituted groups: bond, C3-C20 cycloalkylene, 4-20 membered heterocyclylene, OR11, NR11R12, SO2, NR12SO2, CO or NR12CO; R11 is independently selected from substituted or unsubstituted following groups: C3-C20 cycloalkylene, 4-20-membered heterocyclylene, C3-C20 cycloalkylene C1-C18 alkylene, 4-20-membered heterocyclylene C1-C18 alkylene, C6-C14 aryl or 5-14 membered heteroaryl; R12 is independently selected from the group consisting of substituted or unsubstituted hydrogen, deuterium, C1-C6 alkyl or C3-C6 cycloalkyl;
R1 and R2 are each independently selected from the group consisting of hydrogen, deuterium, halogen, cyano, —(CH2)mR8, —(CH2)m(CH═CH)R8, —(CH2)m(C≡C)R8, —(CH2)mO(CH2)pR8, —(CH2)mSR8, —(CH2)mCOR8, —(CH2)mC(O)OR8, —(CH2)mS(O)qR8, —(CH2)mNR8R9, —(CH2)mC(O)NR8R9, —(CH2)mNR8C(O)R9, —(CH2)mNR8C(O)NR9R10, —(CH2)mS(O)qNR8R9, —(CH2)mNR8S(O)qR9, —(CH2)mNR8S(O)qNR9R10, wherein, H in CH2 can be optionally substituted; R8, R9, R10 are each independently selected from the group of substituted or unsubstituted groups:
hydrogen, C1-C18 alkyl, C1-C18 alkoxy, C3-C20 cycloalkyl, 4-20 membered heterocyclyl, C6-C14 aryl or 5-14 membered heteroaryl; or in —(CH2)mNR8R9, —(CH2)mC(O) NR8R9, —(CH2)mS(O)qNR8R9, R8 and R9 are cyclized with their adjacent N atoms to form a substituted or unsubstituted 4-8-membered heterocyclic group; or in —(CH2)mNR8C(O)R9, —(CH2)mNR8C(O)NR9R10, —(CH2)mNR8S(O)qR9, —(CH2)mNR8S(O)qNR9R10, R8 and R9 are cyclized with their adjacent N atoms to form substituted or unsubstituted 4-8-membered heterocyclyl, or R9 and R10 are cyclized with their adjacent atoms to form a substituted or unsubstituted 4-8-membered heterocyclyl;
R3 is selected from the group consisting of substituted or unsubstituted groups: C3-C18 cycloalkyl, 4-20-membered heterocyclyl, C6-C14 aryl, 5-14-membered heteroaryl;
R4 and R5 are each independently selected from the group consisting of substituted or unsubstituted groups: C1-C6 alkyl, C3-C6 cycloalkyl, 4-6 membered heterocyclic group, ester group, COOH, CONH2, C2-C6 alkene base, C2-C6 alkynyl;
Wherein, the above-mentioned substitution refers to being substituted by one or more groups selected from the group consisting of hydrogen, deuterium, C1-C18 alkyl, deuterated C1-C18 alkyl, halogenated C1-C18 alkyl, halogenated C1-C18 alkylhydroxy, C3-C20 cycloalkyl, C3-C20 cycloalkyl-O—, C1-C18 alkoxy, deuterated C1-C18 alkoxy, halogenated C1-C18 alkoxy, C6-C14 Aryl, 5-14-membered heteroaryl, 4-20-membered heterocyclyl, 4-20-membered heterocyclyl-O—, halogen, oxo C1-C6 alkyl, nitro, hydroxyl, cyano, C2-C6 ester group, C1-C6 amine group, C2-C6 acyl group, C1-C6 amide group, C1-C6 sulfonyl group, C1-C6 sulfonamido group or C1-C6 urea group; wherein, the C1-C18 alkyl group, Deuterated C1-C18 alkyl, halogenated C1-C18 alkyl, halogenated C1-C18 alkyl hydroxyl, C3-C20 cycloalkyl, C3-C20 cycloalkyl-O—, C1-C18 alkoxy, deuterium Substituted C1-C18 alkoxy, halogenated C1-C18 alkoxy, C6-C14 aryl, 5-14-membered heteroaryl, 4-20-membered heterocyclyl, 4-20-membered heterocyclyl-O— also Can be further substituted by one or more Ra, wherein, Ra is selected from: C1-C6 alkyl, deuterated C1-C6 alkyl, halogenated C1-C6 alkyl, halogenated C1-C6 alkyl hydroxyl, C3-C6 Cycloalkyl, C3-C6 cycloalkyl-O—, C1-C6 alkoxy, deuterated C1-C6 alkoxy, halogenated C1-C6 alkoxy, C6-C14 aryl, 5-14 membered hetero Aryl, 4-6 membered heterocyclyl, 4-6 membered heterocyclyl-O—, halogen, oxo C1-C6 alkyl, nitro, hydroxyl, cyano, C2-C6 ester, C1-C6 amine group, C2-C6 amide group, C1-C6 sulfonamido group or C1-C6 urea group; or two substituents located on the same carbon atom together form —(CH2)n— or ═O;
m, n are each independently 0, 1, 2, 3, 4 or 5;
p is 0, 1, 2, 3, 4, or 5;
q is 1 or 2;
The limitation is that when Y is selected from the following group: O, NH or NR7, and Z is a bond, W is a C3-C20 cycloalkylene group or a 4-20-membered heterocyclic group; R1 is not hydrogen, deuterium, halogen, Cyano, R8, O(CH2)pR8, COR8, —C(O)OR8, NR8R9, C(O)NR8R9, —NR8C(O)R9, —NR8C(O)NR9R10.
(h) A pyrimidopyridone derivative, a pharmaceutically acceptable salt thereof, a tautomer or a stereoisomer thereof, wherein the structure of the pyrimidopyridone derivative is as shown in formula (I) as shown: 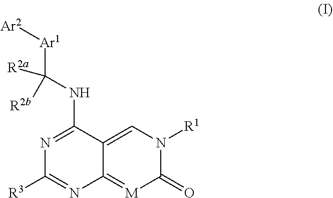
Wherein: R1 is selected from hydrogen or C1-C3 alkyl;
R2 is selected from hydrogen or C1-C3 alkyl, 3-7 membered cycloalkyl, 4-7 membered heterocyclyl, wherein C1-C3 alkyl, 3-7 membered cycloalkyl, 4-7 membered heterocyclyl are any optionally replaced by 1-3 R21;
R21 is selected from C1-C3 alkyl, hydroxyl, halogen, cyano, amino, C1-C3 alkoxy or ═O;
L may be absent or selected from O, NH or N—(C1-C3 alkyl);
R3 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R31;
R31 is selected from C1-C3 alkyl, C1-C3 haloalkyl, hydroxy, halogen, cyano, —NRaRb, C1-C3 alkoxy, ═O, —NHCOR32 or —COR32;
Ra is selected from H, C1-C3 alkyl, C1-C3 haloalkyl or 3-6 membered cycloalkyl;
Rb is selected from H, C1-C3 alkyl, C1-C3 haloalkyl or 3-6 membered cycloalkyl;
R32 is selected from C1-C3 alkyl, C1-C3 haloalkyl, 3-6 membered cycloalkyl or 4-7 membered heterocyclyl;
AR is selected from 6-10-membered aryl or 5-10-membered heteroaryl, wherein the aryl or heteroaryl is optionally substituted by 1-4 R4;
R4 is selected from H, halogen, C1-C3 alkyl, C1-C3 haloalkyl, hydroxy-C1-C3 alkyl, hydroxy-C1-C3 haloalkyl, 3-6 membered cycloalkyl, 4-7 membered heterocyclyl, —ORa, —NRaRb, 6-10-membered aryl or 5-10-membered heteroaryl, wherein the 6-10-membered aryl or 5-10-membered heteroaryl is optionally replaced by 1-4 Rc replaced;
Rc is selected from H, halogen, C1-C3 alkyl, C1-C3 haloalkyl, hydroxy-C1-C3 alkyl, hydroxy-C1-C3 haloalkyl, 3-6 membered cycloalkyl, 4-7 membered heterocyclyl, —ORa, —NRaRb, NRaRb-C1-C4 alkyl, NRaRb-C1-C4 haloalkyl; the heteroatoms in the heterocyclic group or heteroaryl in the formula (I) are 1-3 and are selected from One or more of oxygen, nitrogen and sulfur.
(i) A polycyclic pyridazinone derivative, a pharmaceutically acceptable salt thereof, a tautomer or a stereoisomer thereof,
It is characterized in that, the structure of the polycyclic pyridazinone derivatives is shown in formula (I): 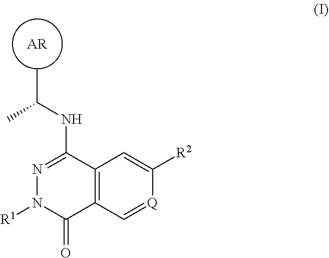
Wherein: R1 is selected from hydrogen or methyl;
R2 is selected from C1-C3 alkyl, —OR21, halogen, 3-7 membered cycloalkyl, 5-7 membered cycloalkenyl, 6-10 membered fused cycloalkyl, 7-10 membered bridged cycloalkyl, 7-10 membered spirocycloalkyl, 4-7 membered heterocyclyl, 5-7 membered heterocycloalkenyl, 6-10 membered fused heterocyclyl, 7-10 membered bridged heterocyclyl, 7-10 membered heterocyclyl A membered spiro heterocyclic group, wherein 3-7 membered cycloalkyl, 5-7 membered cycloalkenyl, 6-10 membered fused cycloalkyl, 7-10 membered bridged cycloalkyl, 7-10 membered spiro Cycloalkyl, 4-7 membered heterocyclyl, 5-7 membered heterocyclenyl, 6-10 membered fused heterocyclyl, 7-10 membered bridged heterocyclyl, 7-10 membered spiro heterocyclyl optionally substituted by 1-3 R22;
R21 is selected from H, C1-C3 alkyl, 3-7 membered cycloalkyl, 4-7 membered heterocyclyl, wherein C1-C3 alkyl, 3-7 membered cycloalkyl, 4-7 membered heterocyclyl are any Optionally replaced by 1-3 R22;
R22 is selected from C1-C3 alkyl, hydroxyl, halogen, cyano, —NRaRb, C1-C3 alkoxy, —C(O)Ra, —C(O)ORa, —OC(O)Ra, —NRbC(O)Ra, —NRbC(O)ORa, —C(O)NRaRb, phenyl, 5-6 membered heteroaryl and ═O, wherein alkyl, alkoxy, phenyl, 5-6 membered heteroaryl group is optionally further substituted with 1-3 halogen, C1-C3 alkyl, hydroxy, cyano, amino and C1-C3 alkoxy;
Ra and Rb are independently selected from H, substituted or unsubstituted C1-C3 alkyl, substituted or unsubstituted 3-6 membered cycloalkyl, or substituted or unsubstituted 4-7 membered heterocyclyl; here “Substituted” means optionally substituted with 1-3 substituents selected from C1-C3 alkyl, hydroxy, halogen, cyano, amino or alkoxy;
Q is selected from N or —CR3;
R3 is selected from H, C1-C3 alkyl, halogen, cyano or —OR21;
AR is selected from 6-10-membered aryl or 5-10-membered heteroaryl, wherein the aryl or heteroaryl is optionally substituted by 1-4 Rc;
Rc is selected from H, halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —OR21, —NRaRb, NRaRb-C1-C4 alkyl, NRaRb-C1-C4 haloalkyl, 6-10-membered aryl or 5-10-membered heteroaryl, wherein 6-10-membered aryl or 5-10 membered heteroaryl is optionally substituted with 1-4 Rd;
Rd is selected from H, halogen, C1-C4 alkyl, C1-C4 haloalkyl, hydroxy-C1-C4 alkyl, hydroxy-C1-C4 haloalkyl, 3-6 membered cycloalkyl, 4-7 membered heterocyclyl, —OR21, —NRaRb, NRaRb-C1-C4 alkyl, NRaRb-C1-C4 haloalkyl;
The heteroatoms in the heterocyclic group, heteroaryl group, heterocyclic alkenyl group, condensed heterocyclic group, bridged heterocyclic group and spiro heterocyclic group in the formula (I) are 1-7 and are selected from oxygen, nitrogen One or more of, sulfur and S(O)m, where m is 1 or 2.
(j) A compound of Formula (I) or Formula (II), 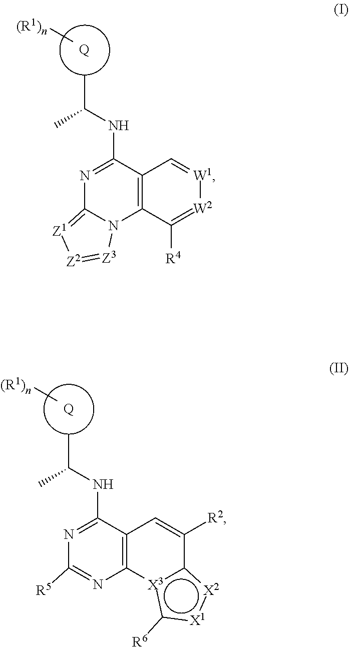
or a pharmaceutically acceptable salt thereof, and/or a tautomer thereof, and/or a stereoisomer thereof,
wherein:
Q at each occurrence is independently a ring selected from phenyl or a 5- or 6-membered heteroaryl group, wherein the heteroaryl group comprises at least one carbon atom and 1-4 additional heteroatoms independently selected from the group consisting of nitrogen, oxygen and sulfur;
Z¹ is N or CR5; Z² is N or CR6;
Z³ is N or CR9;
W¹ is CR2 or NR2;
W² is CR3 when W¹ is CR², and W² is C(O) when
W¹ is NR2; X¹ is N, NR⁷, or CR⁹;
X² is N or CR⁷;
X³ is N or C;
R¹ at each occurrence is independently hydrogen, halogen, C1-6 alkyl, haloC1-6 alkyl, C1-6 alkenyl, C1.5 alkynyl, —NRaRt, OH, C1-6 alkyl-OH, haloC1-6 alkyl-OH, C1-6 alkoxy, haloC1.5 alkoxy, CN, C3.1 cycloalkyl, C3.7 cycloalkyl-OH, C3.7 cycloalkoxy, —S(O)t-C1-oalkyl, —S(O)t-NRaR \ phenyl, or 3-7-membered heterocyclyl, wherein the phenyl and 3-7-membered heterocyclyl are optionally substituted with 1-4 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —S(O)t-C1-6 alkyl, —S(O)t-NRaRb, —NRaRb, or C1.4 alkyl-NRaRt; or two adjacent R1 groups, together with the carbon atoms to which they are attached, form a 5-7-membered carbocyclic or heterocyclic ring optionally substituted with 1-3 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —NR3Rb, C1.4 alkyl-NRaRb, or oxo group (═O);
R² at each occurrence is independently hydrogen, halogen, CN, —ORa, —NRaRh, C1-6 alkyl, haloC1-6 alkyl, C2-6 alkenyl, C2-6 alkynyl, C3.1 cycloalkyl, 3-7-membered heterocylyl, phenyl, or 5-6-membered heteroaryl, wherein each of the C1-6 alkyl, haloC1-6 alkyl, C2-6 alkenyl, C2-6 alkynyl, C3-7 Cycloalkyl, 3-7-membered heterocylyl, phenyl, and 5-6-membered heteroaryl is optionally substituted with 1-5 R8;
R³ at each occurrence is independently hydrogen, halogen, C1-6 alkyl, haloC1-6 alkyl, C1-6 alkoxy, haloC1-6 alkoxy, C1-6 alkyl-OH, CN, C3-1 cycloalkyl, C3.7 cycloalkyl-OH, C3.1 cycloalkoyx, —NH2, —NHC1.4 alkyl, —N(C1.4 alkyl)2, or 3-7-membered cyclic amine;
R⁴ at each occurrence is independently hydrogen, halogen, C1-6 alkyl, haloC1-6 alkyl, C1-6 alkoxy, haloC1-nalkoxy, CN, NH2, C3-7 cycloalkyl or C1-1 cycloalkoxy;
R⁵ at each occurrence is independently hydrogen, C1.4 alkyl, or haloC1.4 alkyl;
R⁶ at each occurrence is independently hydrogen or C1.4 alkyl;
R⁷ at each occurrence is independently hydrogen or C1.4 alkyl;
R⁸ at each occurrence is independently hydrogen, halogen, C1.4 alkyl, haloC1.4 alkyl, C1.4 alkoxy, C2.4 alkenyl, C2.4 alkynyl, C3-7 cycloalkyl, C3-7 cycloalkoxy, 3-7-membered heterocyclyl, phenyl, 5-6-membered heteroaryl, —ORa, —SRa, S(O)tRa, —S(O)t-NRaR \—OC(O)—Ra, —NRaRb, —C(O)Ra, —C(O)ORa, —OC(O)NRaRb2, —C(O)NRaRb, —N(Ra)C(O)ORa, —N(Ra)C(O)Ra, —N(Ra)C(O)NRaR \—N(Ra)C(NRa)NRaRb, —N(Ra)S(O)tNRaRb, —P(═O)(Ra)(Rb), —O—P(═O)(ORa)(ORb), or oxo group (═O);
R⁹ at each occurrence is independently hydrogen or C1.4 alkyl;
Ra and Rb at each occurrence are independently hydrogen, C1-Galkyl, haloC1-Galkyl, C1-Galkyl-OH, C1-6 alkoxy, C3.1 cycloalkyl, 3-7-membered heterocyclyl, C1-6 alkyl-NH2, C1-6 alkyl-NHC1.4 alkyl, C1-6 alkyl-N(C1.4 alkyl)2, or C1-6 alkyl-(3-7-membered cyclic amine); or Ra and Rh, together with the nitrogen atom to which they are attached, form a saturated or unsaturated heterocyclic ring containing from three to seven ring atoms, which ring may optionally contain an additional one or two heteroatoms independently selected from the group consisting of nitrogen, oxygen and sulfur and which ring may be optionally substituted by from one to three substituents independently selected from the group consisting of C1-4 alkyl, phenyl and benzyl;
n at each occurrence is independently 1, 2 or 3, and that each occurrence is independently 1 or 2.
(k) A compound of Formulae (I)-(IV), 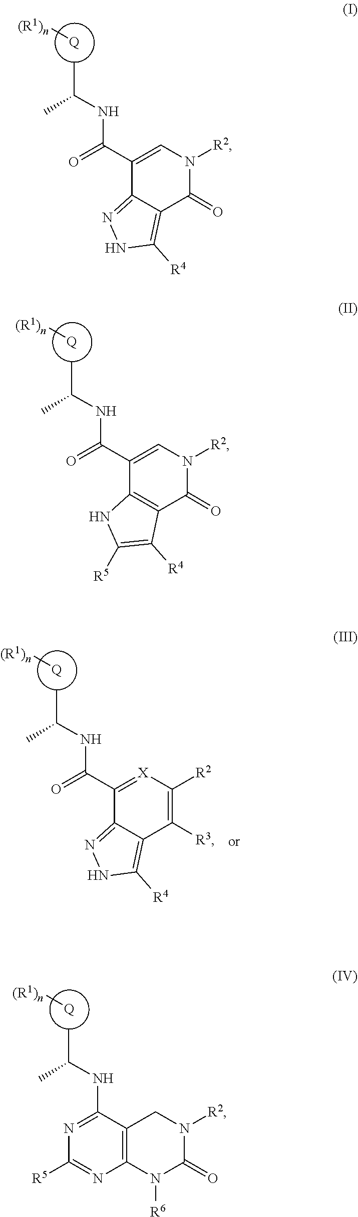
or a pharmaceutically acceptable salt, tautomer or stereoisomer thereof, wherein:
Q at each occurrence is independently a ring selected from phenyl or a 5- or 6-membered heteroaryl group, wherein the heteroaryl group comprises at least one carbon atom and 1-4 additional heteroatoms independently selected from nitrogen, oxygen and sulfur;
X is CH or N;
R1 at each occurrence is independently hydrogen, halogen, C1-6 alkyl, haloC1-6 alkyl, C1-6 alkenyl, C1-6 alkynyl, —NR3Rb, OH, C1-6 alkyl-OH, haloC1-6 alkyl-OH, C1-6 alkoxy, haloC1-6 alkoxy, CN, C3-1 cycloalkyl, C3-7 cycloalkyl-OH, C3-7 Cycloalkoxy, —S(O)t-C1-6 alkyl, —S(O)t-NR3R \ phenyl, or 3-7-membered heterocyclyl, wherein the phenyl and 3-7-membered heterocyclyl are optionally substituted with 1-4 substituents independently selected from C1-4 alkyl, haloC1-4 alkyl, C1-4 alkoxy, haloC1-4 alkoxy, C1-4 alkyl-OH, haloC1-4 alkyl-OH, OH, halogen, CN, —S(O)t-C1-6 alkyl, —S(O)t-NRaRh, —NR3Rh, and C1-4 alkyl-NRaRh, or two adjacent R1 groups, together with the carbon atoms to which they are attached, form a 5-7-membered carbocyclic or heterocyclic ring optionally substituted with 1-3 substituents independently selected from C1.4 alkyl, haloC1.4 alkyl, C1.4 alkoxy, haloC1.4 alkoxy, C1.4 alkyl-OH, haloC1.4 alkyl-OH, OH, halogen, CN, —NRaRh, C1.
4 alkyl-NR3Rh, and oxo group (═O);
R² at each occurrence is independently hydrogen, halogen, CN, —OR³, —NR3Rh, C1-6 alkyl, haloC1.6 alkyl, C2-6 alkenyl, C2-6 alkynyl, C3.7 cycloalkyl, 3-7-membered heterocylyl, phenyl, or 5-6-membered heteroaryl, wherein each of the C1-6 alkyl, haloC1-6 alkyl, C2-6 alkenyl, C2-6 alkynyl, C3.7 cycloalkyl, 3-7-membered heterocylyl, phenyl, and 5-6-membered heteroaryl is optionally substituted with 1-5 R⁸;
R³ at each occurrence is independently hydrogen, halogen, C1-6 alkyl, haloC1-6 alkyl, C1-6 alkoxy, haloC1-Galkoxy, C1-Galkyl-OH, CN, C3.1 cycloalkyl, C3.1 cycloalkyl-OH, C3.1 cycloalkoyx, —NH2, —NHC1.4 alkyl, —N(C1.4 alkyl)2, or 3-7-membered cyclic amine;
R⁴ at each occurrence is independently hydrogen, halogen, C1-6 alkyl, haloC1-6 alkyl, C1-6 alkoxy, haloC1-6 alkoxy, CN, NH2, C3.7 cycloalkyl or C3.1 cycloalkoxy;
R⁵ at each occurrence is independently hydrogen, C1.4 alkyl, or haloC1.4 alkyl;
R⁶ at each occurrence is independently hydrogen, C1-oalkyl, haloC1-oalkyl, or C1-1 cycloalkyl;
R⁸ at each occurrence is independently hydrogen, halogen, C1.4 alkyl, haloC1.4 alkyl, C1.4 alkoxy, C2.4 alkenyl, C2.4 alkynyl, C3.7 cycloalkyl, C3.7 cycloalkoxy, 3-7-membered heterocyclyl, phenyl, 5-6-membered heteroaryl, —OR³, —SR³, S(O)tR³, —S(O)tNR³Rh, —OC(O)—R³, —NR³Rh, —C(O)R³, —C(O)OR³, —OC(O)NR³Rb2, —C(O)NR³Rb, —N(R³)C(O)OR³, —N(R³)C(O)R³, —N(R³)C(O)NR³R \—N(R³)C(NR³)NR³R \—N(R³)S(O)tNR³R \—P(═O)(R³)(Rb), —O—P(═O)(OR³)(ORb), or oxo group (═O);
R³ and Rb at each occurrence are independently hydrogen, C1-6 alkyl, haloC1-6 alkyl, C1-6 alkyl-OH, C1-6 alkoxy, C3-7 cycloalkyl, 3-7-membered heterocyclyl, C1-6 alkyl-NH2, C1-6 alkyl-NHC1.4 alkyl, C1-6 alkyl-N(C1.4 alkyl)2, or C1-6 alkyl-(3-7-membered cyclic amine), wherein each of the foregoing groups may be optionally substituted by one to three substituents independently selected from the group consisting of C1-4 alkyl, haloC1.4 alkyl, halogen, OH, NH2, C1.4 alkoxy, haloC1.4 alkoxy, CN, and —C(O)C1.4 alkyl; or Ra and Rh, together with the nitrogen atom to which they are attached, form a saturated or unsaturated heterocyclic ring containing from three to seven ring atoms, which ring may optionally contain an additional one or two heteroatoms independently selected from the group consisting of nitrogen, oxygen and sulfur and may be optionally substituted by from one to three substituents independently selected from the group consisting of C1-4 alkyl, —C(O)C1.4 alkyl, phenyl and benzyl;
n at each occurrence is independently 1, 2 or 3, and that each occurrence is independently 1 or 2.
(I) a Compound Having a Structure of Formula (I), Formula (II), or Formula (III): 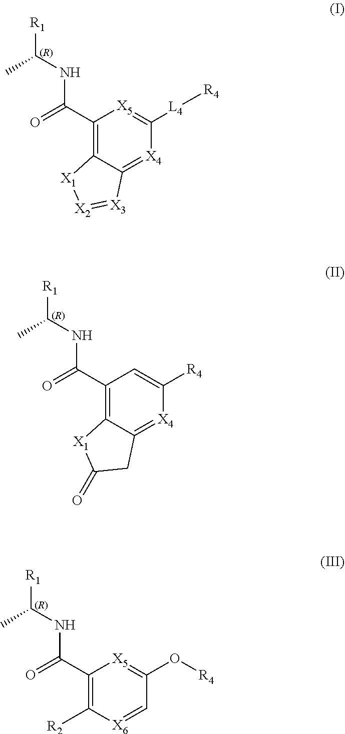
or a pharmaceutically acceptable salt, solvate, stereoisomer, prodrug, or tautomer thereof, wherein:
X1 is NH or S;
X2 is CH or N;
X3 is CH or N;
X4 is CR³ or N
X5 is CH or N;
X6 is CH or N;
R1 is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R2 is selected from the group consisting of H, —NH—C1-6 alkyl, and —NH2;
R3 is selected from the group consisting of H, —O—C1-6 alkyl, and —O—C1-6 heteroalkyl;
L4 is a bond or O; and
R4 is selected from the group consisting of H, C1-6 alkyl, 3-14 membered cycloalkyl, 3-14 membered cycloalkenyl, 3-14 membered heterocyclyl, 6-10 membered aryl, and 5-10 membered heteroaryl; wherein each C1-6 alkyl, 3-14 membered cycloalkyl, 3-14 membered cycloalkenyl, 3-14 membered heterocyclyl, 6-10 membered aryl, and 5-10 membered heteroaryl is optionally substituted with one or more C1-6 alkyl, -R⁴a, —OR⁴a, —O—C1-6 alkyl-R4a, ═O, halogen, —C(O)R⁴a, —C(O)OR⁴a, —C(O)NR⁴bR⁴c, —NR⁴bC(O)R⁴c, —CN, ═NR⁴a, —NR⁴bR4c, —SO2R⁴a, 3-6 membered cycloalkyl optionally substituted with R4a, 3-7 membered heterocyclyl optionally substituted with R⁴a, 6-10 membered aryl optionally substituted with R4a, or 5-10 membered heteroaryl optionally substituted with R4a;
wherein R4a is H, C1-6 alkyl, C1-6 haloalkyl, —C(O)R4b, —C(O)NR4bR4c, ═O, 3-6 membered cycloalkyl, 6-10 membered aryl optionally substituted with —OR4b, —CN, ═N-3-6 membered cycloalkyl, 3-7 membered heterocyclyl, —(CH2)rOCH3, or —(CH2)rOH, wherein r is 1, 2, or 3;
wherein each R4b is independently H, C1-6 alkyl; and
wherein each R4c is independently H or C1-6 alkyl.
(m) A compound of formula I: 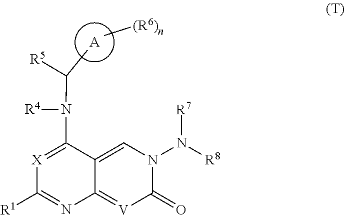
a pharmaceutically acceptable salt or a stereoisomer thereof, wherein:
ring A is 3-12 membered carbocyclyl, 3-12 membered heterocyclyl, 6-10 membered aryl, or 5-10 membered heteroaryl;
R¹ is hydrogen, halogen, C1-6 alkyl, or C3-6 cycloalkyl, wherein said C1-6 alkyl or
C3-6 cycloalkyl represented by R¹ is optionally substituted by one to more groups selected from halogen and —OH;
V is N or CR²; wherein
R² is hydrogen, halogen, —CN, C1-6 alkyl, C2-6 alkenyl, C2-6 alkynyl, —OR²U, —NR²aR²h, —C(O)R²U, —C(O)OR²a, —C(O)NR²aR²h, —SO2R²U, —SO2NR²aR²h, —P(O)R²aR²h, —NR² aC(O)R²h, —NR² aC(O)OR²h, —NR² aSO2R²h, —NR² aSO2NR²bR2c, 3-12 membered carbocyclyl, 3-12 membered heterocyclyl, 6-10 membered aryl, or 5-10 membered heteroaryl; wherein
said C1-6 alkyl, C2-6 alkenyl, C2-6 alkynyl, 3-12 membered carbocyclyl, 3-12 membered heterocyclyl, 6-10 membered aryl, or 5-10 membered heteroaryl represented by R² is optionally substituted by one or more R²d; wherein
R²a, R²b, and R²c are independently selected from the group consisting of hydrogen, C1-6 alkyl, 3-12 membered carbocyclyl, 3-12 membered heterocyclyl,
6-10 membered aryl, and 5-10 membered heteroaryl; or R² and R²h or R²h and R²c together with the N or P atom to which they are attached form 4-12 membered heterocyclyl or 5-10 membered heteroaryl;
wherein said C1-6 alkyl, carbocyclyl,
heterocyclyl, aryl, or heteroaryl represented by R²U, R²b, or R²c or in the group represented by R²U, R²h, or R²c are optionally substituted with one or more R²d; wherein
R²d, in each occurrence, is hydrogen, halogen, oxo, —CN, C1-6 alkyl, C1-6haloalkyl, —OR²—\ NR²eR²r, —C(O)R²\—C(O)OR²\—C(O)NR²eR²r, —SO2R²\ —SO2NR²eR²r, —P(O)R²eR²r, —NR²eC(O)R²r, —NR²eC(O)OR²r, —NR²eSO2R²r, —NR²eSO2NR²rR²g, 3-12 membered carbocyclyl, 3-12 membered heterocyclyl, 6-10 membered aryl, or 5-10 membered heteroaryl;
R²e, R²r, and R²g are independently selected from the group consisting of hydrogen and C1-6 alkyl;
X is N or CR³;
R³ is hydrogen, halogen, or C1-3 alkyl;
R⁴ is hydrogen or C1-6 alkyl;
R⁵ is hydrogen, C1-6 alkyl, 3-6 membered monocyclic carbocyclyl, or 4-6 membered monocyclic heterocyclyl; wherein said C1-6 alkyl, 3-5 membered monocyclic carbocyclyl, or 4-6 membered monocyclic heterocyclyl represented by R⁵ is optionally substituted with one or more groups selected from halogen and —OH;
R⁶ is hydrogen, —OH, halogen, —CN, oxo, C1-6 alkyl, C1-6 alkoxy, —SO2R⁶\ —SO2NR⁶aR⁶b, —P(O)R⁶aR⁶b, —C(O)NR⁶aR⁶b, —NR⁶ aC(O)R⁶\—NR⁶ aC(O)NR⁶aR⁶b, —(CH2)sNR⁶aR⁶h, —O(CH2)rNR⁶aR⁶h, 3-12 membered carbocyclyl, 3-12 membered heterocyclyl, 6-10 membered aryl, 5-membered heteroaryl; wherein said C1-6 alkyl, C1-6 alkoxy, 3-12 membered carbocyclyl, 3-12 membered heterocyclyl, 6-membered aryl, or 5-10 membered heteroaryl represented by R⁶ is optionally substituted by one to more R⁶c; wherein R⁶a and R⁶b are independently hydrogen or C1-6 alkyl, or R⁶a and R⁶b together with the N or P atom to which they are attached form 4-7 membered heterocyclyl;
s is an integral from 0 to 3;
t is an integral from 2 to 4;
R⁶C, in each occurrence, is hydrogen, —OH, halogen, —CN, oxo, C1-6 alkyl, C1-6 alkoxy, C3-6 cycloalkyl, —NR⁶aR⁶—\ SO2R⁶\—SO2NR⁶aR⁶h, —C(O)NR⁶aR⁶h, —P(O)R⁶aR⁶b, —NR⁶ aC(O)R⁶U, —NR⁶ aC(O)NR⁶aR⁶b, —(CH2),NR⁶aR⁶b, or —O(CH2)rNR⁶aR⁶b; wherein said C1-6 alkyl or C3-6 Cycloalkyl represented by R⁶c is optionally substituted with one to more groups selected from halogen, —OH and —NR⁶aR⁶b;
R⁷ and R⁸ are independently hydrogen, C1-6 alkyl, C3-6 alkenyl, C3-6 alkynyl, C2-6 alkoxy, 3-12 membered carbocyclyl, 3-12 membered heterocyclyl, 6-10 membered aryl, or 5-10 membered heteroaryl; wherein said C1-6 alkyl, C3-6 alkenyl, C3-6 alkynyl, C2-6 alkoxy, 3-12 membered carbocyclyl, 3-12 membered heterocyclyl, 6-10 membered aryl, or 5-10 membered heteroaryl represented by R⁷ or R⁸ is optionally substituted by one or more R⁷a; or
R⁷ and R⁸ together with the N atom to which they are attached form 4-12 membered heterocyclyl or 5-10 membered heteroaryl; wherein said 4-12 membered heterocyclyl or 5-10 membered heteroaryl is optionally substituted with one or more R⁷b;
R⁷a is hydrogen, halogen, —CN, C1-6 alkyl, —OR⁷C, —NR⁷ cR⁷ ct, —C(O)R⁷C, —C(O)OR⁷C, —C(O)NR⁷ cR⁷d, —SO2R⁷C, —P(O)R⁷ cR⁷d, —SO2NR⁷ cR⁷d, —NR⁷ cC(O)R⁷d, —NR⁷ cC(O)OR⁷ ct, —NR⁷ cSO2R⁷ ct, —NR⁷ cSO2NR⁷ ctR⁷3-12 membered carbocyclyl, 3-12 membered heterocyclyl, 6-10 membered aryl, or 5-10 membered heteroaryl, wherein said C1-6 alkyl, 3-12 membered carbocyclyl, 3-12 membered heterocyclyl, 6-10 membered aryl, or 5-10 membered heteroaryl represented by R⁷a is optionally substituted by one or more R⁷f;
R⁷b is hydrogen, halogen, —CN, oxo, C1-6 alkyl, C2-6 alkenyl, C2-6 alkynyl, C1-6 alkoxy, —OR⁷c, —NR⁷ cR⁷ ct, —C(O)R⁷C, —C(O)OR⁷c, —C(O)NR⁷ cR⁷ ct, —SO2R⁷C, —P(O)R⁷ cR⁷ ct, —SO2NR⁷ cR⁷ ct, —NR⁷ cC(O)R⁷ ct, —NR⁷ cC(O)OR⁷ ct, —NR⁷ cSO2R¹ ct, —NR⁷ cSO2NR⁷dR^{7\} 3-12 membered carbocyclyl, 3-12 membered heterocyclyl, 6-10 membered aryl, or 5-10 membered heteroaryl, wherein C1-6 alkyl, C2-6 alkenyl, C2-6 alkynyl, C1-6 alkoxy, 3-12 membered carbocyclyl, 3-12 membered heterocyclyl, or 5-10 membered heteroaryl represented by R⁷b is optionally substituted by one or more R1r;
R⁷C, R⁷ ct, and R⁷e are independently selected from the group consisting of hydrogen, C1-6 alkyl, 3-12 membered carbocyclyl, 4-12 membered heterocyclyl, 6-10 membered aryl, and 5-10 membered heteroaryl; or R⁷c and R⁷d together with the
N or P atom to which they are attached form 4-12 membered heterocyclyl or 5-10 membered heteroaryl; wherein said C1-6 alkyl, carbocyclyl, heterocyclyl, aryl, or heteroaryl represented by R⁷C, R⁷ ct, or R⁷e or in the group represented by R⁷C, R⁷ ct, or R⁷e is optionally substituted with one or more R⁷f;
R⁷f, in each occurrence, is hydrogen, halogen, —CN, or —OH; and
n is 0, 1, 2, or 3;
wherein said heterocyclyl comprises 1-4 heteroatoms selected from O, N, and S; and said heteroaryl comprises 1-4 heteroatoms selected from O, N, and S.
(n) The compound according to formula (II) 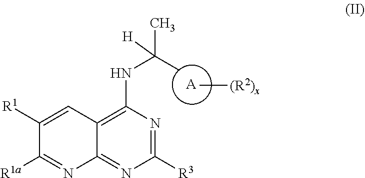
wherein
A is phenyl;
is selected from halogen, 5 to 10 membered mono or bicyclic heterocycloalkyl or heterocycloalkenyl with one or 2 nitrogen as heteroatoms and substituted by —CH3, —C(═O)—CH3 or —NH—C(═O)—CH3,
R¹a is selected from hydrogen, —CH3, CF3 or —OCH3;
R² is selected from hydrogen, halogen or C1-6-alkyl optionally one or more time substituted by halogen and/or hydroxyl;
x is selected from 1 or 2 and is selected from hydrogen or —CH3; or a stereoisomer, a tautomer, an N-oxide, a hydrate, a solvate, or a salt thereof, or a mixture of same.
(o) A compound of Formula (I): 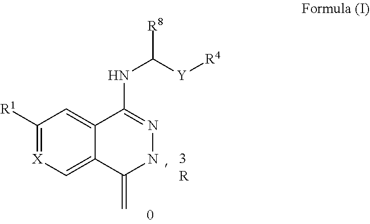
or a pharmaceutically acceptable salt thereof,
wherein:
R¹ is hydrogen, hydroxyl, C1-C6 alkyl, alkoxy, —N(R⁶)2, —NR⁶C(O)R⁶, —C(O)N(R⁶)2, —SO2 alkyl, —SO2NR⁶ alkyl, cycloalkyl, -Q-heterocyclyl, aryl, or heteroaryl, wherein the cycloalkyl, the heterocyclyl, the aryl, or the heteroaryl are each optionally substituted with one or more R²;
each Q is independently a bond, 0 or NR⁶;
X is N or CR⁹; with the proviso that when X is N, R¹ is not hydroxyl;
each R² is independently hydroxy, halogen, cyano, hydroxyalkyl, haloalkyl, alkoxy, —N(R⁶)2, —SO2 alkyl, —NR⁶C(O)C1-C3 alkyl, —C(O)cycloalkyl, —C(O)heterocyclyl or aryl, wherein the cycloalkyl, the heterocyclyl or the aryl are each optionally substituted with one or more R⁹;
R³ is hydrogen, C1-C3 alkyl, C1-C3 haloalkyl, or cycloalkyl;
Y is a bond or heteroarylene;
R⁴ is aryl or heteroaryl, each optionally substituted with one or more R¹;
each R⁵ is independently hydroxy, halogen, cyano, hydroxyalkyl, alkoxy, C1-C4 alkyl, haloalkyl, —N(R⁶)2, -L-N(R⁶)2 or —SO2 alkyl;
L is C1-C3 alkylene;
each R⁶ is independently hydrogen, C1-C3 alkyl, haloalkyl or cycloalkyl;
R⁷ is hydrogen, cyano or alkoxy;
R⁸ is C1-C2 alkyl or halo-C1-C2 alkyl; and
each R⁹ is independently C1-C3 alkyl or haloalkyl.

2. The use according to claim 1 wherein the SOS1 inhibitor is selected from: 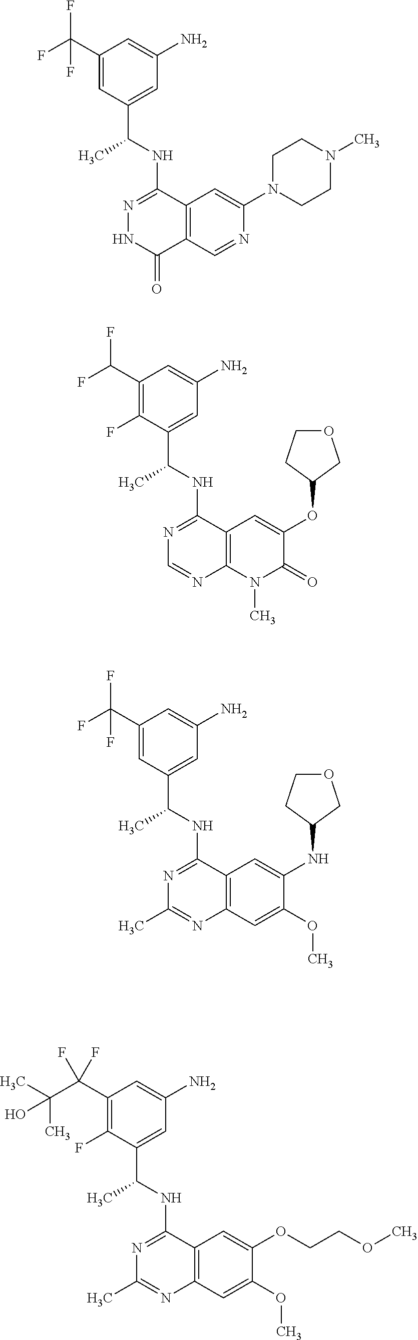 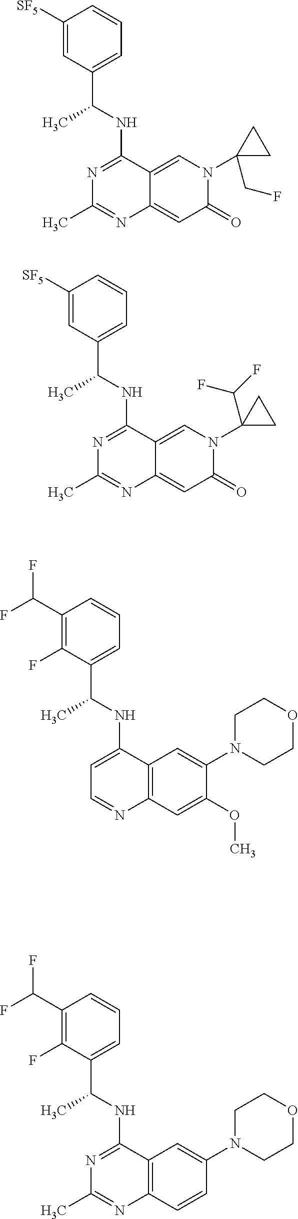 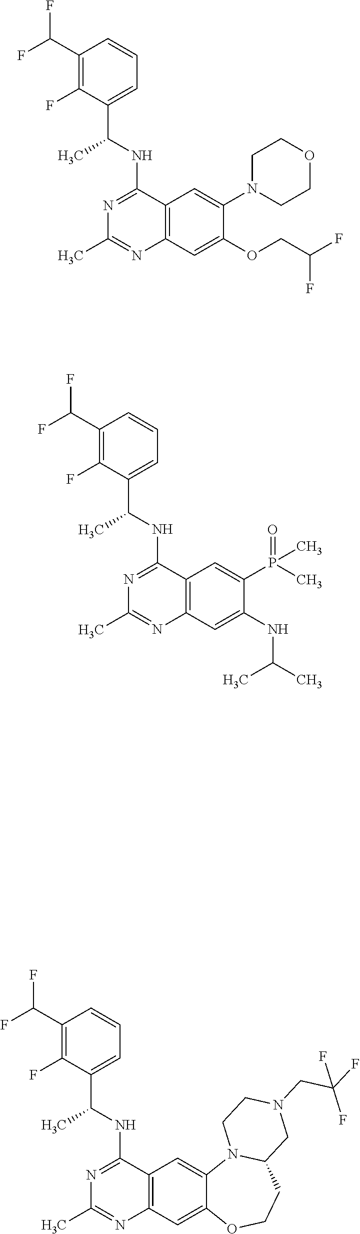 [SMILES: Cc1nc(N[C@H](C)c2cccc(C(F)F)c2F)c2cc(N3CCOCC3)c(OCC(F)F)cc2n1 ; Cc1nc(N[C@H](C)c2cccc(C(F)F)c2F)c2cc(P(C)(C)=O)c(NC(C)C)cc2n1 ; Cc1nc(N[C@H](C)c2cccc(C(F)F)c2F)c2cc3c(cc2n1)OCC[C@H]1CN(CC(F)(F)F)CCN31] 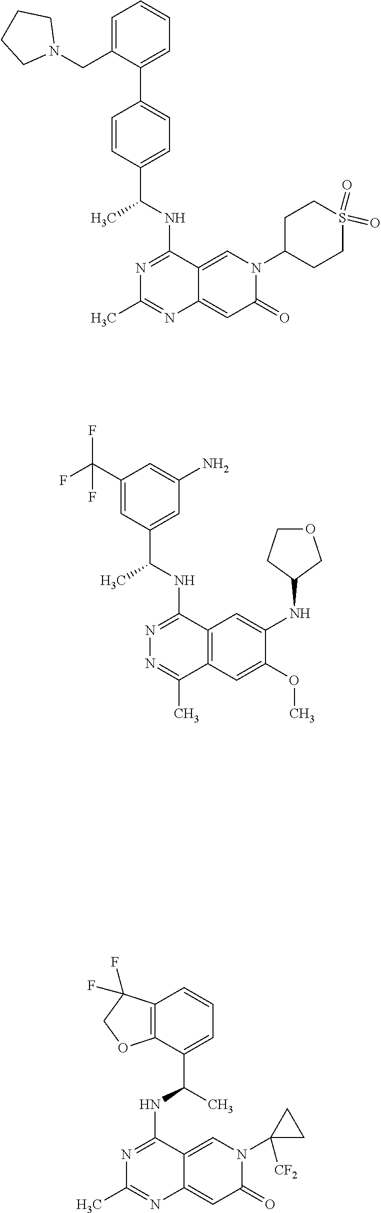
4-[[(1R)-1-(3,3-difluoro-2H-1-benzofuran-7-yl)ethyl]amino]-6-[1-(difluoromethyl)cyclopropyl]-2-methylpyrido[4,3-d]pyrimidin-7-one. [chemical structure]
